(19) United States
(12) Patent Application Publication
(10) Pub. No.: US 2012/0304991 A1
(43) Pub. Date: Dec. 6, 2012

(54) DRUG PRODUCTS AND DRY POWDER INHALERS WITH MULTIPLE RESERVOIRS

(76) Inventors: Mikhail Gotliboym, Scotch Plains, NJ (US); Annaniy Berenshteyn, Ocean, NJ (US); Aleksandr Zuyev, Denville, NJ (US); Scott Brown, Princeton, NJ (US); Robert L. Berger, Bound Brook, NJ (US)

(21) Appl. No.: 13/509,746

(22) PCT Filed: Nov. 9, 2010
(86) PCT No.: PCT/US10/55995

§ 371 Date: Aug. 9, 2012

Related U.S. Application Data
(60) Provisional application No. 61/261,140, filed on Nov. 13, 2009.

(51) Int. Cl. A61M 15/00

(52) U.S. Cl. 128/203.15

(57) ABSTRACT

Various embodiments of the present invention provide drug products and dry powder inhalers and powder dispensers with multiple reservoirs. Several embodiments provide a drug product comprising a dry powder inhaler and at least one dose of at least one active pharmaceutical agent; wherein the dry powder inhaler comprises at least two reservoirs. Other embodiments provide for a powder dispenser which includes a first powder reservoir having at least one first opening, and a second powder reservoir having at least one second outlet opening, the second outlet opening being spaced from the first outlet opening.

### FIELD OF THE INVENTION

This invention relates to dry powder inhalers and more particularly, to dry powder inhalers with multiple reservoirs.

### BACKGROUND

Various devices have been used in order to dispense an inhaled metered dose of active pharmaceutical agents such as, including pressurized aerosol devices, nebulizers, pump inhalators and the like. There is growing demand for powder dispensing devices which can dispense metered doses of powdered medicament. With such devices, the powder is withdrawn by inhalation so there is less need to be concerned with synchronizing release of medication with the exact start of inspiration to insure quality of the product delivery. Additionally, dry powders may be more stable than the liquid compositions that may be found in other inhaler device forms.

The particles containing the APA that leave the DPI are desirably within a particular size range that target a specific area of the lung. If the particles containing the APA are too large, they may not enter the respiratory tract, but instead, will be deposited in the mouth or pharynx and possibly enter the digestive tract.

Current dispensers may have one reservoir that holds the powder in the form of agglomerates that contain an active pharmaceutical agent. As the device is actuated, the reservoir will release a dose of agglomerates that contains the appropriate dose of the APA. After the device is actuated, the consumer inhales to force the agglomerates to be carried through inhaler flow channels and break up into a micronized powder. This micronized powder will desirably deliver a consistent dose of the APA to the targeted lung area of the consumer.

Current designs for dry powder inhalers are described in U.S. Pat. No. 6,240,918, U.S. Pat. No. 5,829,434, U.S. Pat. No. 5,394,868, U.S. Pat. No. 5,687,710. Desirably, the DPI will be a device that is easy to operate for consumer which means that it should not too big or cumbersome so that the DPI is easy for the consumer to use. Thus, DPIs are desirably small and easy to manipulate for the consumer. The total delivered dose of APA of current DPIs may be limited due to the fact that only a certain total amount of dry powder may be dispensed from the current DPIs due to desirable size requirements of the DPIs. Also, the capacity of the powder carrying channel may not be able to accommodate and sufficiently de-agglomerate large dosing loads due to a limited capacity to deliver and de-agglomerate the powder.

Some APAs may not be able to manufactured in one agglomerate. For instance, the processing parameters of a dry powder for a specific APA may require the APA to be prepared separately from another APA or two APAs may be incompatible with each other, e.g. the actives may cause chemical degradation or particle size changes to another APA. Thus, in order to ensure consistent content uniformity of a dose, dosing of more than one APA from a single DPI may require individual agglomerates to be located in different reservoirs.

Thus, it would be desirable to be able to increase the dose capacity of DPIs and also to accommodate powders that contain two or more APAs that may not be compatible with each other or are manufactured as separate agglomerates.

### SUMMARY

A powder dispenser is provided herein which includes a first powder reservoir having at least one first opening, and a second powder reservoir having at least one second outlet opening, the second outlet opening being spaced from the first outlet opening. In addition, the dispenser includes a metering dose plate having a first metered dose hole and a second metered dose hole, the metered dose holes being configured to each hold a predetermined amount of powder. The metering dose plate is disposed adjacent to the first and second outlet openings with the metering dose plate, relative to the outlet openings, being reversibly movable between a first position and a second position. With the metering dose plate moving from the first position to the second position relative to the outlet openings, the first metered dose hole passes below the first outlet opening and the second metered dose hole passes below the second outlet opening. Further, the first metered dose hole defines a first fixed path as the metering dose plate moves reversibly between the first and second positions relative to the outlet openings, and the second metered dose hole defines a second fixed path as the metering dose plate moves reversibly between the first and second positions relative to the outlet openings. The first fixed path is spaced from the second fixed path such that the first metered dose hole does not overlap the second path during movement of the metering dose plate and such that the second metered dose hole does not overlap the first path during movement of the metering dose plate. Advantageously, with the subject invention, at least two different powders may be accommodated by a dispenser with the powders being delivered in a controlled manner. During preparation of the doses in the metering dose plate, the two paths of travel of the dose holes are kept separated to limit cross-contamination therebetween.

Further embodiments provide a drug product comprising a dry powder inhaler and at least one dose of at least one active pharmaceutical agent; wherein the dry powder inhaler comprises at least two reservoirs comprising the at least one dose. The at least two reservoirs may separately comprise different active pharmaceutical agents. Those different active pharmaceutical agents may be incompatible with each other, e.g. cause chemical degradation or particle size changes. When the dry powder inhaler is actuated, the at least one dose is emitted simultaneously from the at least two reservoirs.

Other embodiments of the present invention provide a drug product comprising a dry powder inhaler and at least one dose of at least one active pharmaceutical agent; wherein the dry powder inhaler comprises at least two reservoirs separately comprising different active pharmaceutical agents and the at least two reservoirs comprises the at least one dose that are emitted simultaneously from the at least two reservoirs when the dry powder inhaler is actuated. The different active pharmaceutical agents may be incompatible with each other, e.g. cause chemical degradation or particle size changes.

Still further embodiments provide a powder dispenser comprising a first powder reservoir having at least one first outlet opening; a second powder reservoir having at least one second outlet opening, the second outlet opening being spaced from the first inlet opening.

These and other features of the invention will be better understood through a study of the following detailed description and accompanying drawings.

### BRIEF DESCRIPTION OF THE DRAWINGS

FIGS. 1-2 are perspective views of a metered powder dose dispenser formed in accordance with the subject invention;

FIG. 3 is an exploded view of a metered powder dose dispenser formed in accordance with the subject invention;

FIG. 4 is a cross-sectional view taken along line 4-4 of FIG. 1;

FIGS. 5-7 show a reservoir body useable with the subject invention;

FIGS. 8a and 8b are cross-sectional views taken along lines 8a-8a and 8b-8b, respectively, of FIG. 7;

FIG. 9 shows a reservoir plug useable with the subject invention;

FIGS. 10-12 show a driving body useable with the subject invention;

FIG. 13 is a cross-sectional view taken along line 13-13 of FIG. 12;

FIG. 14 is a cross-sectional view taken along line 14-14 of FIG. 11;

FIG. 15 shows an assembly of a reservoir body, a driving body and a reservoir plug useable with the subject invention;

FIGS. 16-19 show a metering dose plate useable with the subject invention;

FIG. 20 is a schematic showing movement of the dose holes of a metering dose plate over a range of motion in accordance with the subject invention;

FIG. 21 shows a metering dose plate having powder retainers extending over dose holes of a metering dose plate useable with the subject invention;

FIGS. 22-25 show a base useable with the subject invention;

FIG. 26 is a cross-sectional view taken along line 26-26 of FIG. 22;

FIGS. 27-31 show a lower spring retainer useable with the subject invention;

FIGS. 32-33 show a support plate useable with the subject invention;

FIG. 34 shows an alternative arrangement of a powder retainer useable with the subject invention;

FIGS. 35-36 show an adapter useable with the subject invention;

FIGS. 37-39 show a swirl nozzle useable with the subject invention;

FIG. 40 shows an assembly of a mouthpiece and swirl nozzle useable with the subject invention;

FIGS. 41 and 44 show a mouthpiece useable with the subject invention;

FIGS. 42 and 43 are cross-sectional views taken along line 42-42 and line 43-43, respectively, of FIG. 41;

FIGS. 45-47 show a closure cap useable with the subject invention;

FIGS. 48A-48B and FIGS. 49A-49B show the operation of a metered powder dose dispenser in accordance with the subject invention;

FIGS. 50-53 show a continuous counter ring useable with the subject invention;

FIGS. 54-57 show an intermittent counter ring useable with the subject invention;

FIGS. 58-62 show a spring-biased pawl assembly useable with the subject invention;

FIGS. 63-66 show an alternate spring-biased pawl assembly useable with the subject invention; and,

FIGS. 67-71 show a further alternate of a spring-biased pawl assembly useable with the subject invention.

### DETAILED DESCRIPTION OF SPECIFIC EMBODIMENTS

Various embodiments of the present invention are useable in connection with dispensing large doses of powder and with doses of different powders, for example, in combination therapy where at least two active pharmaceutical agents are used. This is especially useful for at least two active pharmaceutical agents that may not be compatible with each other, e.g. APAs that may cause one or more of the APAs to degrade when in the presence of each other. Doses of 25 to 1600 μg of APAs are possible. The doses may include one or more additional substances beyond the at least one APA, such as carrier(s) and/or secondary agent(s). For example, a 400 mg dose may contain 3 mg of active agent; a 200 mg dose may contain 1.5 mg of active agent; and, a 100 mg dose may contain 0.75 mg of active agent. U.S. Pat. No. 6,240,918 describes various features of a powder dispenser which may be utilized in conjunction with the subject invention. U.S. Pat. No. 6,240,918 is incorporated by reference in its entirety herein. In addition, U.S. Pat. Nos. 5,829,434; 5,687,710; and, 5,394,868 describe various features of powder dispensers useable with the subject invention. U.S. Pat. Nos. 5,829,434; 5,687,710; and, 5,394,868 are each incorporated by reference herein in their respective entireties.

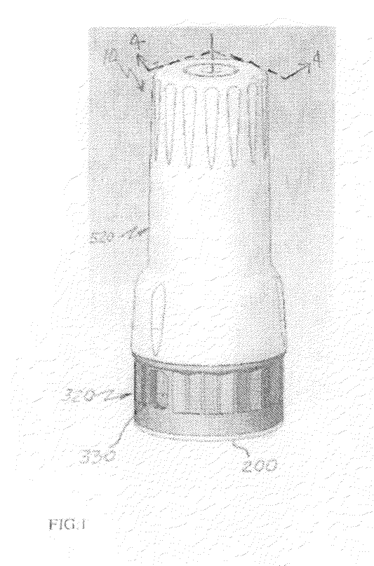

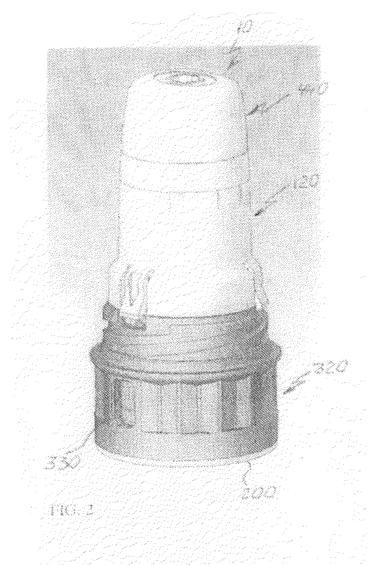

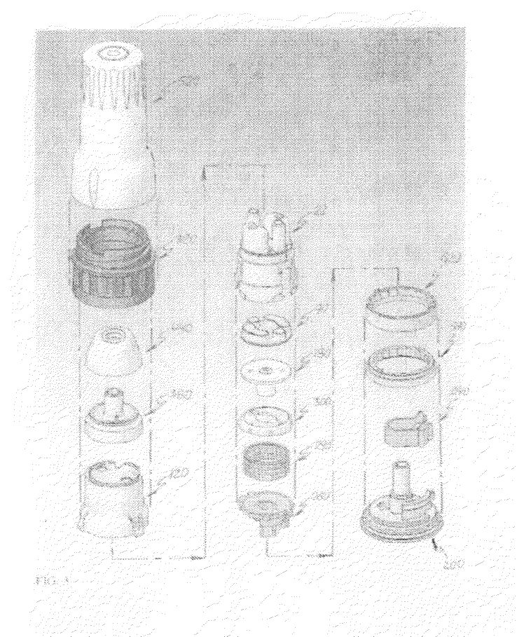

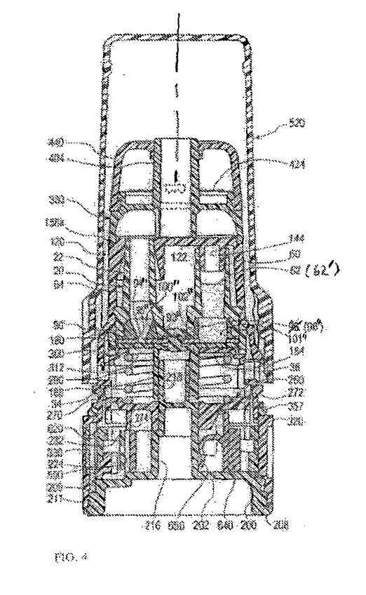

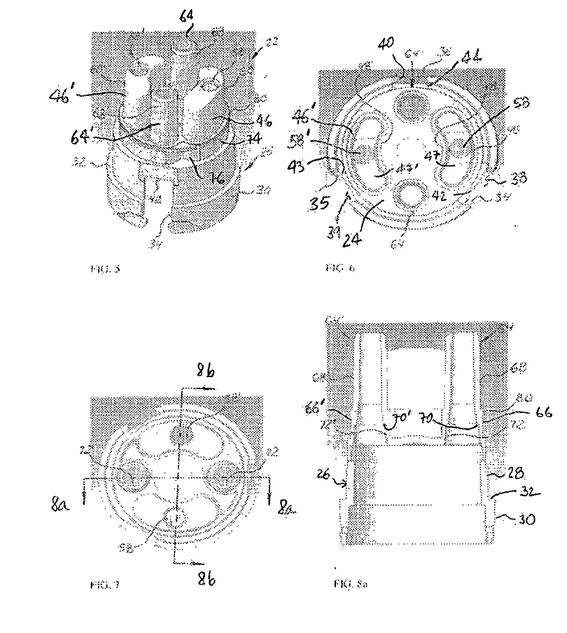

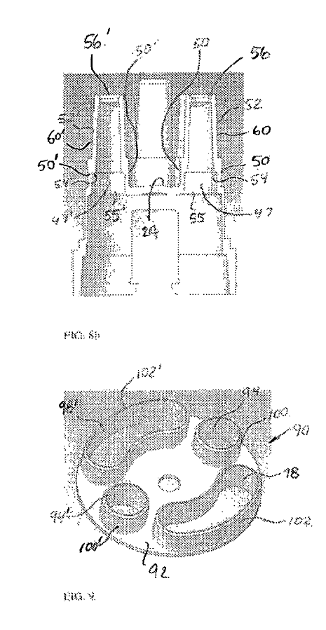

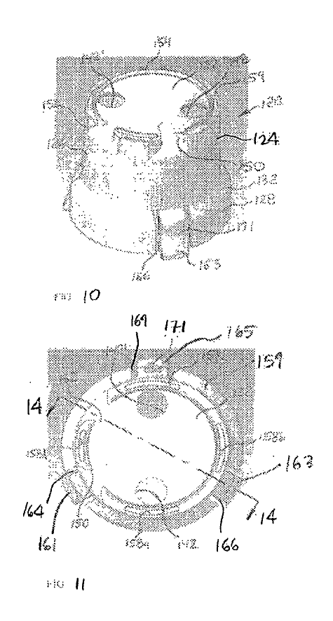

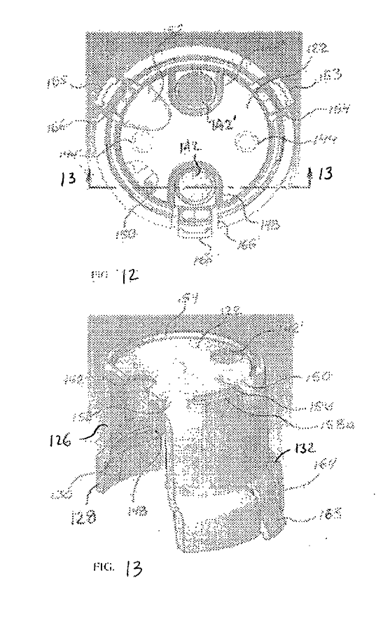

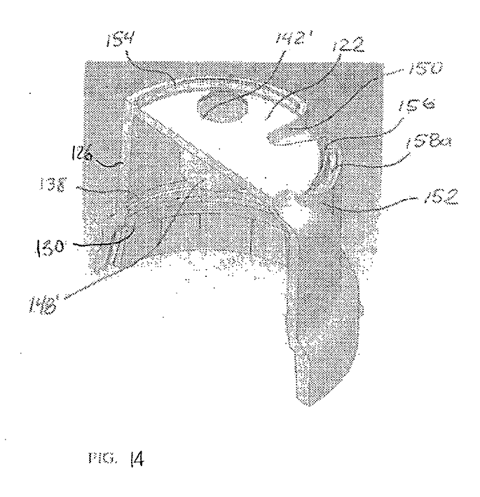

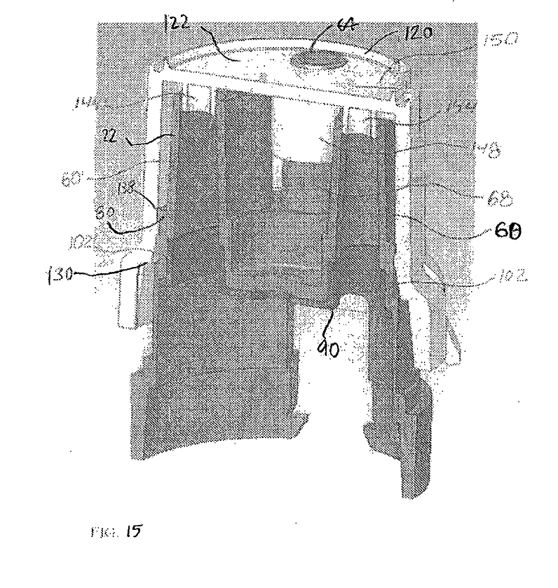

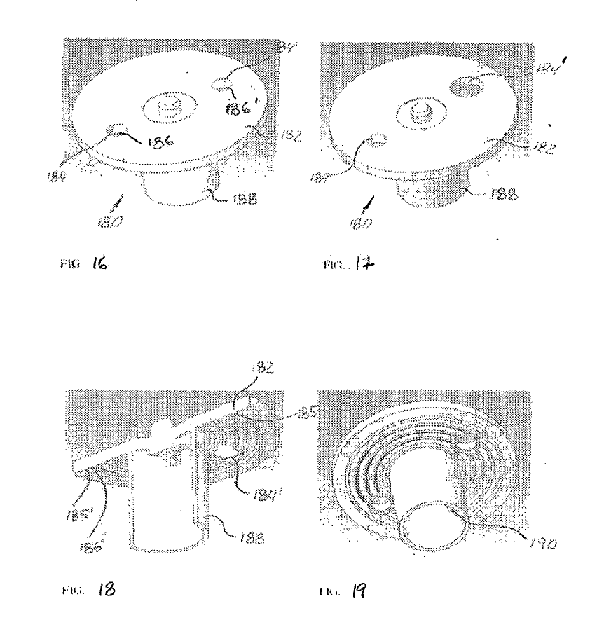

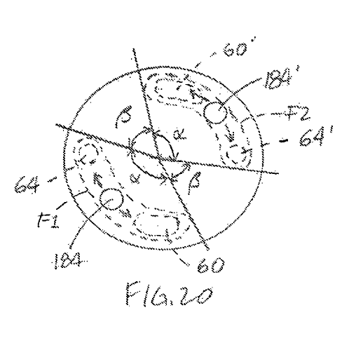

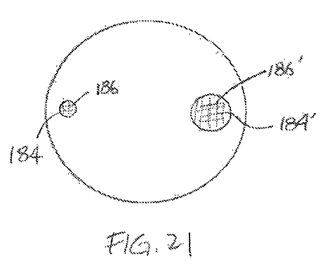

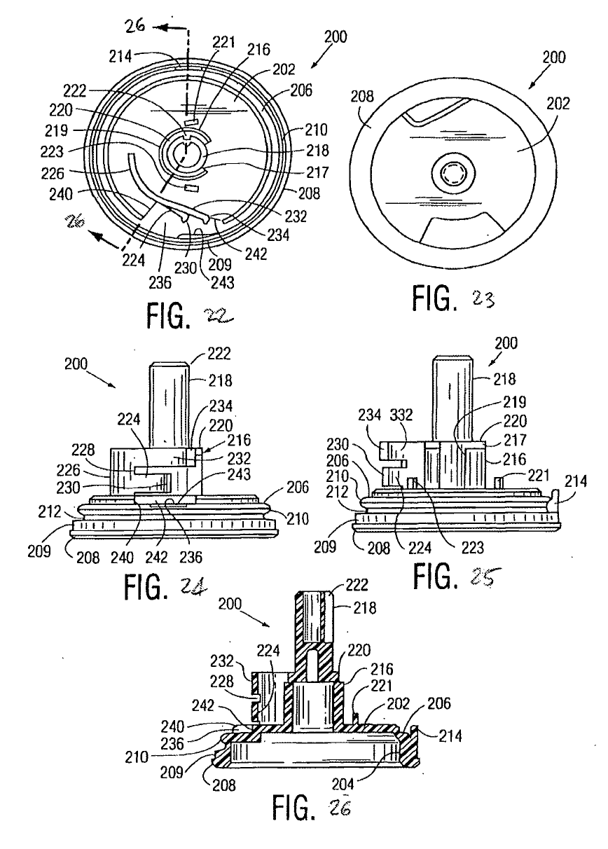

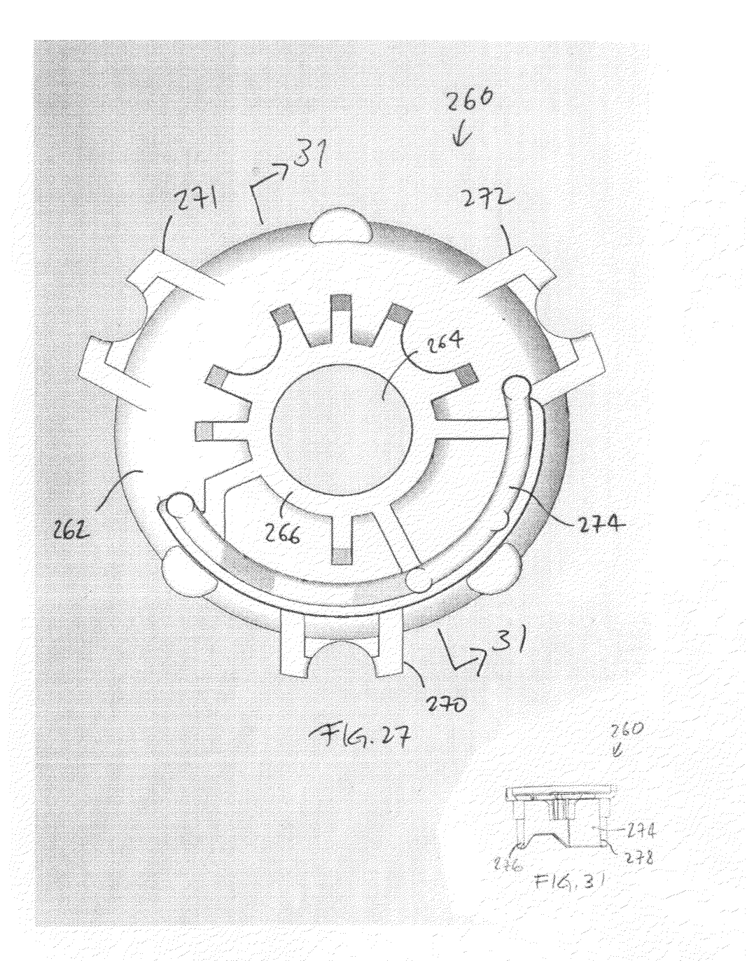

Referring to the drawings in detail, and initially to FIGS. 1-4 thereof, a metered powder dose dispenser 10 according to the present invention includes a powder housing 20 for holding a supply of powdered material to be dispensed, and for supplying metered doses of the powder to a user.

Powder housing 20 is comprised of a reservoir body 22, a reservoir plug 90 and a driving body 120, each preferably being formed as a single molded plastic piece.

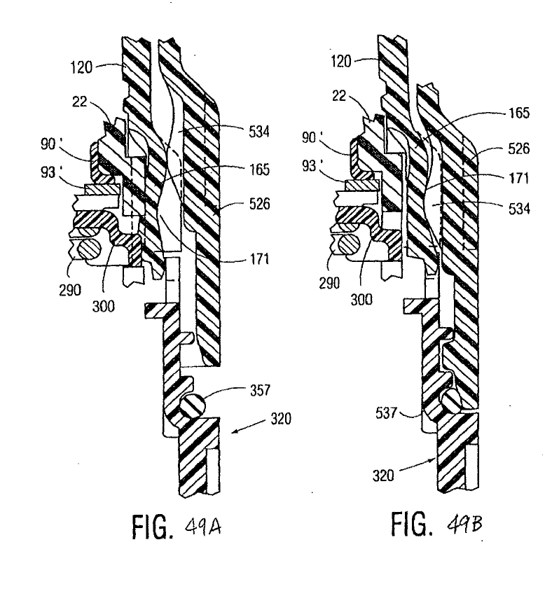

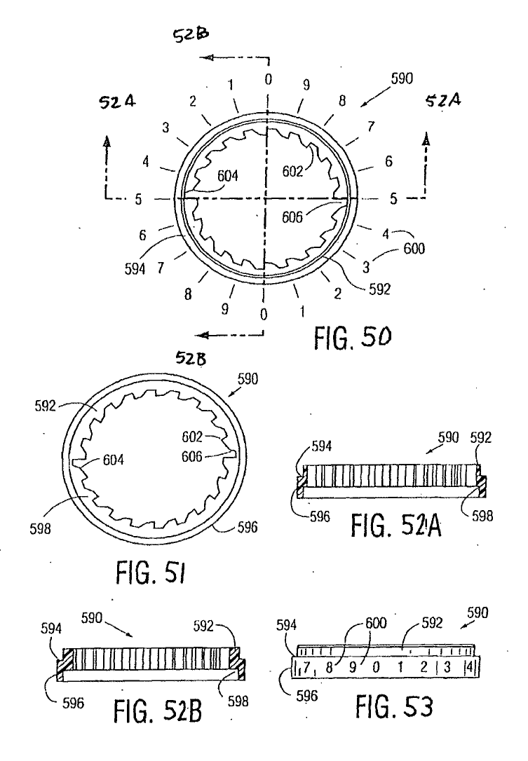

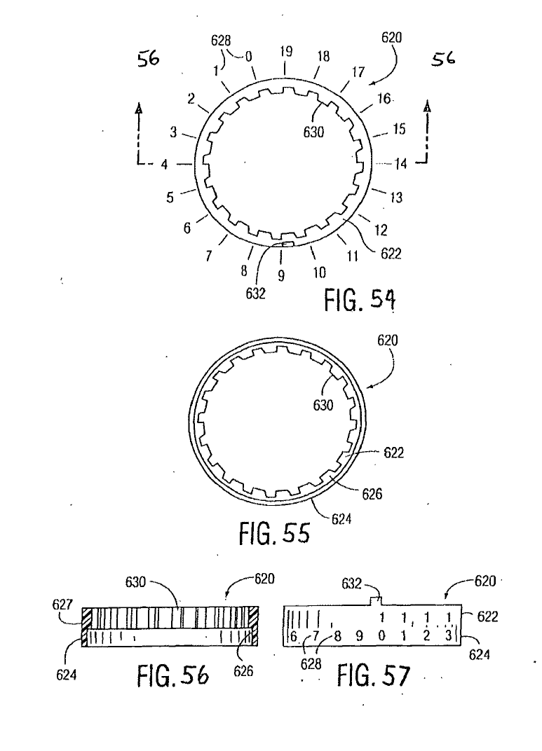

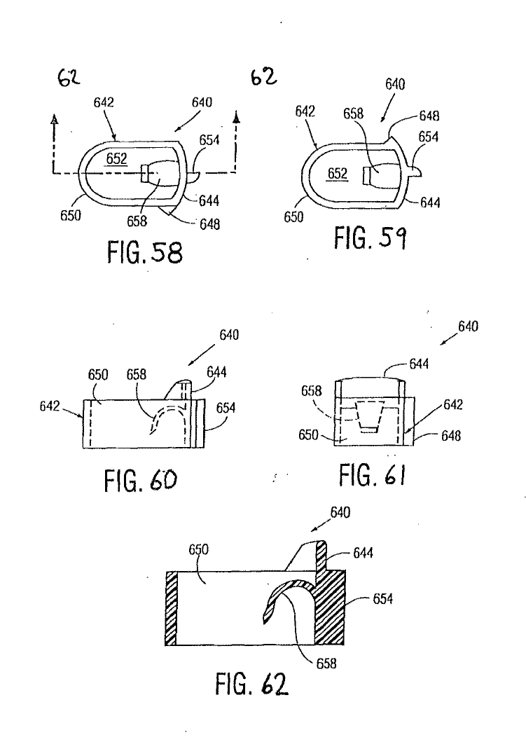

Referring to FIGS. 3-8b, reservoir body 22 includes a circular top wall 24 having an annular skirt 26 extending downwardly from the periphery of circular top wall 24. Annular skirt 26 includes an upper annular skirt section 28 with its upper end extending downwardly from the periphery of circular top wall 24, and a lower annular skirt section 30 extending downwardly from the lower end of upper annular skirt section 28. Lower annular skirt section 30 has an inner and outer diameter greater than the inner and outer diameters, respectively, of upper annular skirt section 28. Accordingly, an outer annular shoulder 32 is formed at the upper end of lower annular skirt section 30.

Spaced apart axially extending drive slots 34, 35 and 36 are formed in annular skirt 26. Preferably, the drive slots 34, 35, 36 have centers equally spaced about the circumference of the annular skirt 26 (e.g., spaced apart at approximately 120°). Further, it is preferred that the drive slot 36, which is axially aligned with the venturi conduits 64, 64′ described below, have a shorter circumferential length than the drive slots 34, 35. Of course, the present invention is not limited to this particular configuration. Drive slots 34, 35, 36 are open at their lower ends 38, 39, 40, respectively, and extend upwardly entirely through lower annular skirt portion 30 and partially through upper annular skirt portion 28. Thus, drive slots 34, 35, 36 have closed upper ends which define seating edges 42, 43, 44.

Powder housing 20 includes arcuate manifolds 46, 46′ formed on the upper surface of circular top wall 24, at a peripheral position offset from the center thereof. Each of the manifolds 46, 46′ includes respectively an arcuate chamber 47, 47′ extending circumferentially for an arcuate length about a peripheral portion of circular top wall 24 and which is defined by a surrounding chamber wall 48, 48′. Specifically, each chamber wall 48, 48′ is formed by a lower chamber wall portion 50, 50′ extending upwardly from circular top wall 24 and an upper chamber wall portion 52, 52′ extending upwardly from the upper end of lower chamber wall portion 50, 50′. The shapes of wall portions 50, 50′ and 52, 52′ are substantially identical, but with the inner dimensions of upper wall portion 52, 52′ being less than the inner dimensions of lower wall portion 50, 50′. As a result, a shoulder 54, 54′ is formed at the lower end of each upper chamber wall portion 52, 52′.

Circular top wall 24 includes openings 55, 55′ of the same shape and dimensions as lower chamber wall portions 50, 50′ of manifolds 46, 46′ and in alignment respectively with the lower end of each lower chamber wall portion 50, 50′. The upper end of each manifold 46, 46′ and particularly each upper chamber wall portion 52, 52′ is closed by a manifold top wall 56, 56′ which is angled downwardly from the center thereof and which has an opening 58, 58′ at the center thereof.

The manifolds 46, 46′ each define a powder supply conduit 60, 60′ formed as a supply holder. The upper end of each powder supply conduit 60, 60′ is open at the openings 58, 58′. Each powder supply conduit 60, 60′ is respectively normally filled with powder 62, 62′ for inhalation. As used herein, the terms “powdered medicaments” and “powder” include micronized powder, spheronized powder, micro-encapsulated powder, powder agglomerates and the like, and are used interchangeably with these terms herein. As will be appreciated by those skilled in the art, the powders 62, 62′ may be different powders or the same powders. By using different powders 62, 62′, combination therapy with different drug combinations may be achieved. In addition, with the powders 62, 62′ being the same powder, a relatively large dose of a single powder may be delivered to a user.

Frusto-conical inhalation venturi conduits 64, 64′ are also formed on circular top wall 24 substantially parallel to powder supply conduits 60, 60′ and axially offset from the central axis of circular top wall 24. The center axis of powder supply conduit 60 and the center axis of venturi conduit 64 lie on a circle having a center coincident with the center of circular top wall 24, so as to be positioned at a peripheral portion of circular top wall M. Likewise, the center axis of powder supply conduit 60′ and the center axis of venturi conduit 64′ lie on a circle having a center coincident with the center of circular top wall 24, so as to be positioned at a peripheral portion of circular top wall 24. Preferably, the center axes of powder supply conduits 60, 60′ and venturi conduits 64, 64′ lie on the same circle having a center coincident with the center of circular top wall 24.

It is preferred that supply conduits 60, 60′ and venturi conduits 64, 64′ be equally spaced about the center of circular top wall 24 (e.g., the conduits 60, 60′ and 64, 64′ are approximately 90° spaced apart). It is further preferred that supply conduits 60, 60′ and venturi conduits 64, 64′ be arranged in alternating fashion.

Specifically, venturi conduits 64, 64′ are each formed by a lower venturi conduit section 66, 66′ and an upper venturi conduit section 68, 68′ axially aligned therewith, each reducing in inner diameter from a lower end thereof to an upper end thereof. The upper end of each upper venturi conduit section 68, 68′ is open, and each upper venturi conduit section 68, 68′ has a smaller diameter than the corresponding lower venturi conduit section 66, 66′ so that an inner annular shoulder 70, 70′ is formed at the lower edge of upper venturi conduit section 68, 68′. Circular top wall 24 includes further openings 72, 72′ of the same shape and dimensions as the corresponding lower end of lower venturi conduit section 66, 66′ and in alignment therewith.

A peripheral securing wall 74 extends generally about a circular arc on a peripheral portion of circular top wall 24, in surrounding relation to lower chamber wall portions 50, 50′ and lower venturi conduit sections 66, 66′. One or more gaps 76 are provided in securing wall 74 at a position between conduits 60 and 64′. Further, a radially extending annular lip 80 may extend outwardly from the upper end of securing wall 74.

As will be understood from the description hereinafter, it is necessary that the surface for engaging with the metering dose plate, as described below, be as smooth as possible, that is, with very few undulations therein. The lower surface of the circular top wall 24 could be utilized. However, this is difficult to achieve when molding reservoir body 22 as a single piece. Therefore, to overcome this problem, a reservoir plug 90 is provided, as shown in FIG. 9.

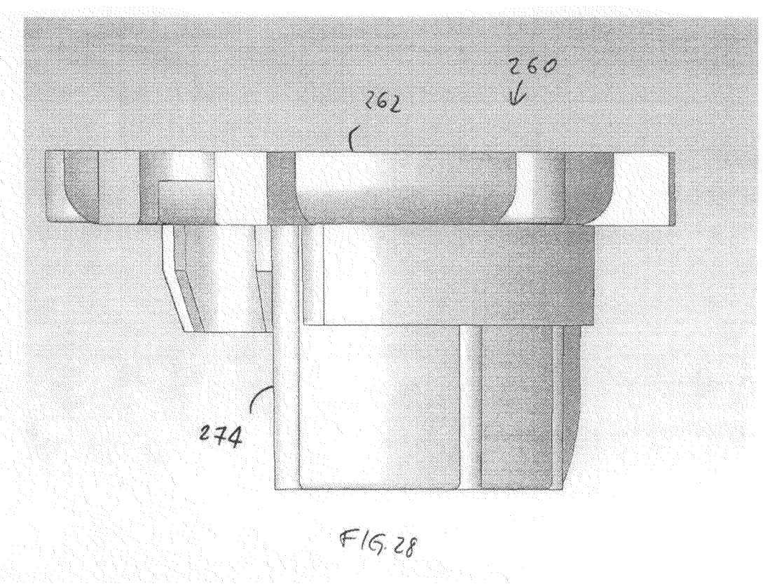

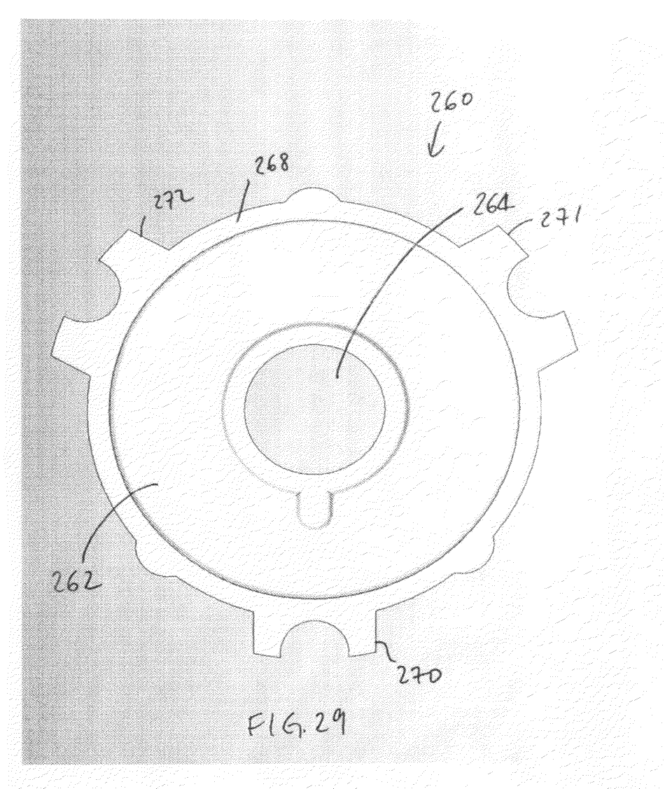

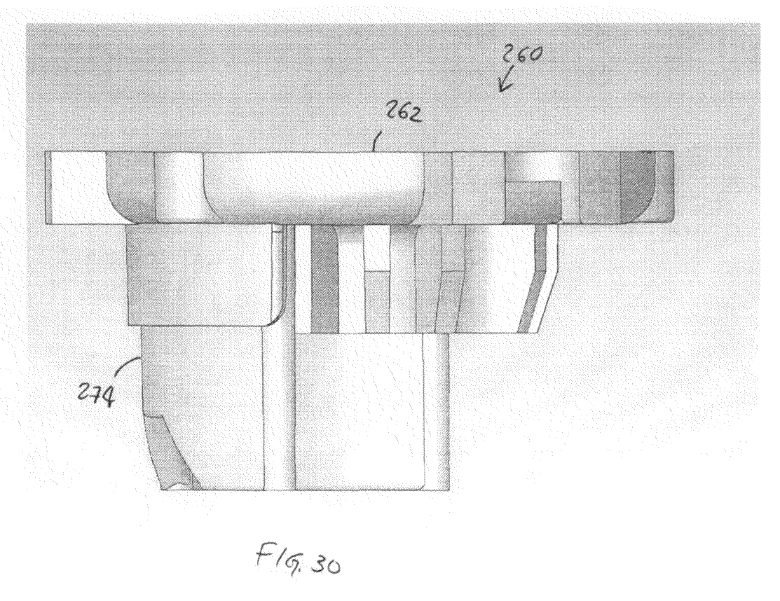

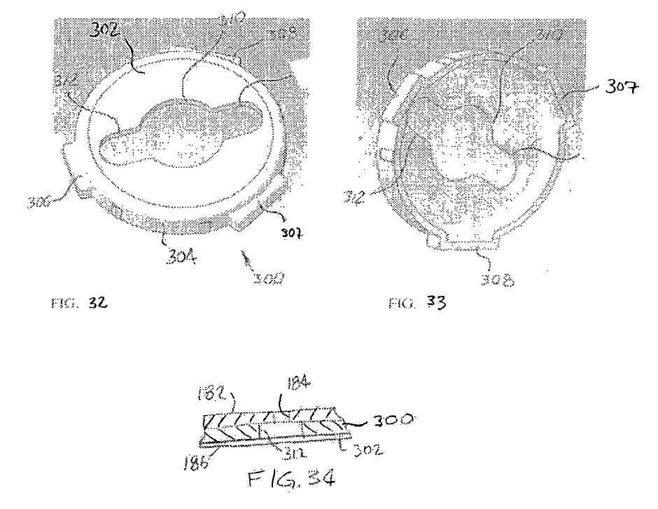

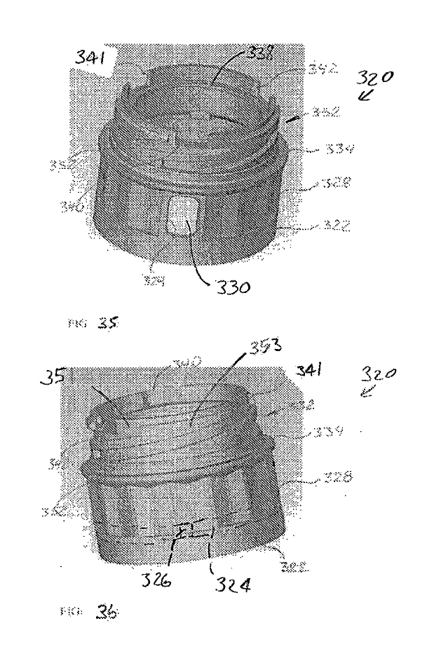

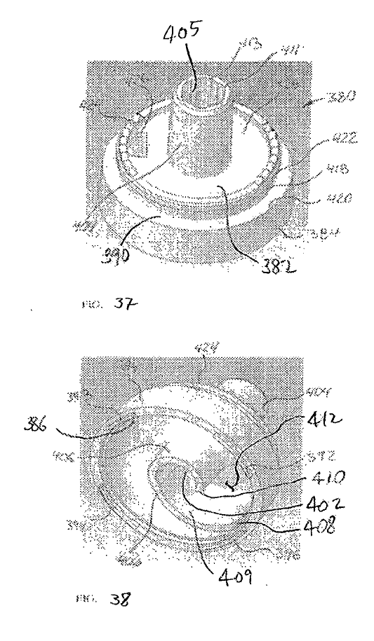

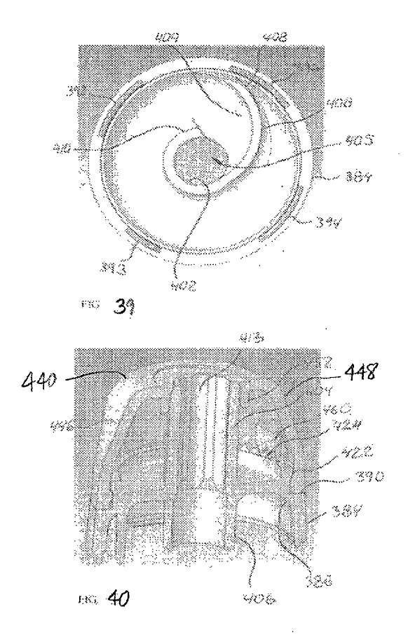

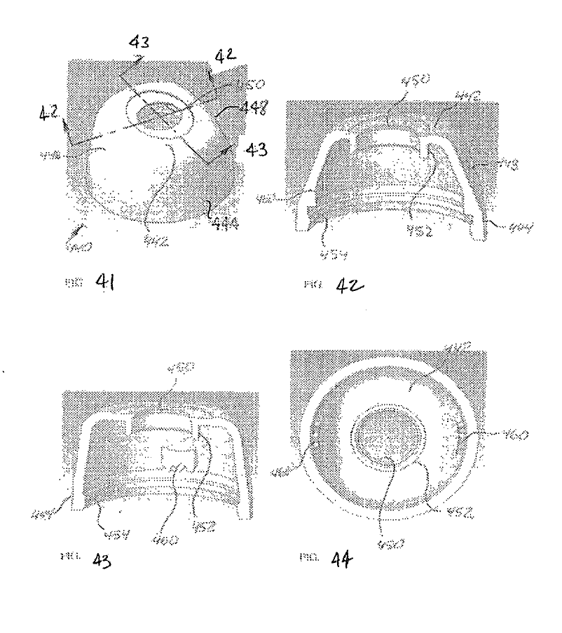

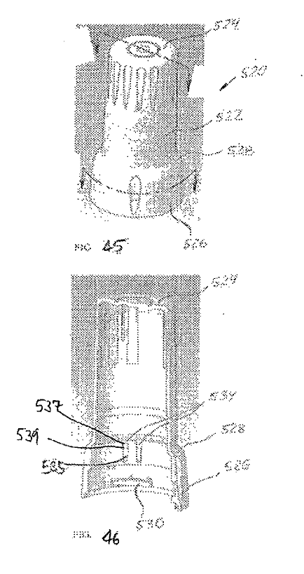

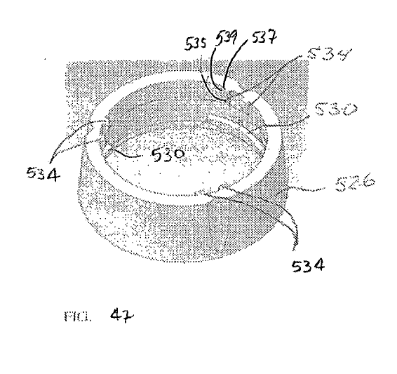

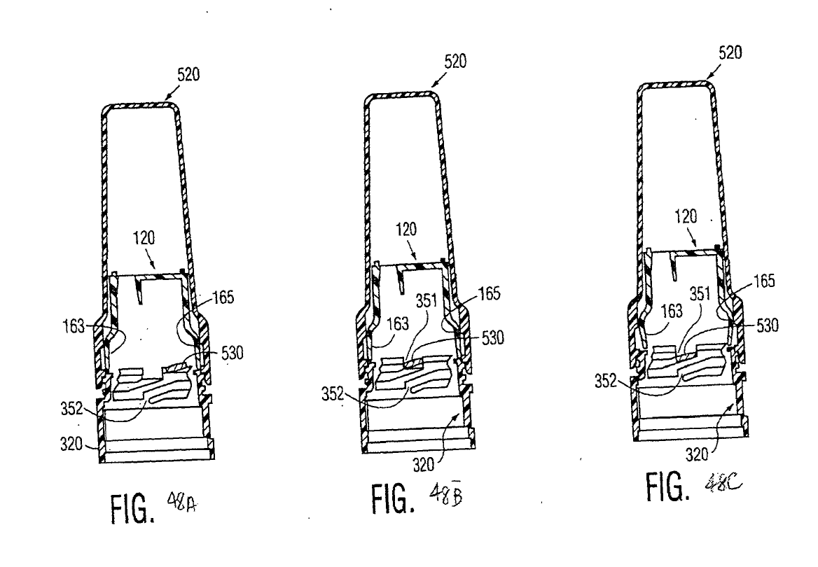

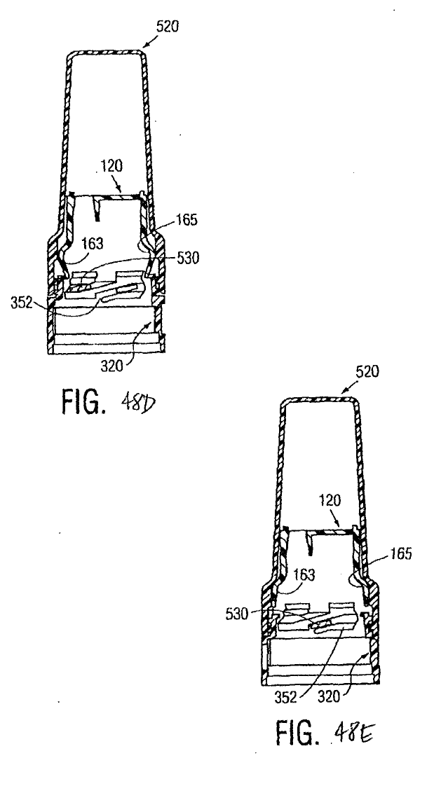

Specifically, reservoir plug 90 includes a thin circular plate 92 which can be molded, because of the thinness of plate 92, to have a very smooth lower surface with no undulations. The outer diameter of circular plate 92 is substantially equal to the inner diameter of upper annular skirt portion 28 so that reservoir plug 90 can be fit therein, as shown in FIG. 4. In such condition, the lower surface of circular plate 92 effectively is flush with seating edges 42, 43, 44 of drive slots 34, 35, 36.

Circular plate 92 has two circular holes 94, 94′ and two substantially oval holes 98, 98′, all preferably having centers extending along an imaginary circle centered at the center of plate 92.

Circular plug conduits 100, 100′ formed on the upper surface of circular plate 92 have surrounding relation to circular holes 94, 94′, respectively. Conduits 100, 100′ are each open at its upper and lower ends and have an outside diameter and a height substantially equal to the inside diameter and height, respectively, of corresponding lower venturi conduit sections 66, 66′ and an inside diameter equal to the inside diameter of corresponding upper venturi conduit sections 68, 68′. Thus, when reservoir plug 90 is inserted within upper annular skirt section 28, plug conduits 100, 100′ fit snugly within lower venturi conduit sections 66, 66′ and the inner surfaces of plug conduits 100, 100′ each forms a smooth continuation of the corresponding inner surface of upper venturi conduit section 68, 68′. In such condition, the upper edge of each plug conduit 100, 100′ abuts against corresponding annular shoulder 70, 70′ so that no gap is formed between plug conduit 100, 100′ and corresponding upper venturi conduit section 68, 68′.

Arcuate plug conduits 102, 102′ are formed on the upper surface of circular plate 92 in surrounding relation to corresponding substantially oval holes 98, 98′. Each plug conduit 102, 102′ has the same shape as corresponding lower chamber wall portion 50, 50′ of manifolds 46, 46′. Each plug conduit 102, 102′ is open at its upper and lower ends and has an outside shape and dimensions substantially equal to the inside shape, height and dimensions, respectively, of corresponding lower chamber wall portion 50, 50′ inside shape and dimensions equal to the inside shape and dimensions of corresponding upper chamber wall portion 52, 52′. Thus, as shown in FIG. 15, when reservoir plug 90 is inserted within upper annular skirt section 28, plug conduits 102, 102′ fit snugly within lower chamber wall portions 50, 50′ and the inner surfaces of plug conduits 102, 102′ each forms a smooth continuation of the corresponding inner surface of upper chamber wall portion 52, 52′. In such condition, the upper edge of each plug conduit 102, 102′ abuts against corresponding shoulder 54, 54′ so that no gap is formed between plug conduit 102, 102′ and corresponding upper chamber wall portion 52, 52′.

Although the outer surfaces of plug conduits 100, 100′ and 102, 102′ are discussed above as being smooth, it will be appreciated that such outer surfaces can be formed with ribs.

As an alternative embodiment of reservoir plug 90, a reservoir plug 90′ is shown in the cross-sectional FIG. 4, in which elements corresponding to those of reservoir plug 90 are identified by the same reference numerals, with a double prime (″) appended thereto.

As shown, at least one plug conduit 100″ has an inner diameter with a frusto-conical configuration that tapers from an upper end to a lower end thereof, to provide a venturi effect. In addition, the inner diameter of at least one arcuate plug conduit 102″ may be greater than the inner diameter of upper chamber wall portion 52, 52′. Further, to better ensure a smooth lower surface, a thin flat, circular metal plate 93″ of electropolished stainless steel is secured to the lower surface of reservoir plug 90″. In such case, plate 92″ has openings 101″ of the same dimensions as arcuate plug conduits 102″, while oval holes 98″ are provided in metal plate 93″. Of course, metal plate 93″ has further circular openings 95″ coincident with circular holes 94″ of circular plate 92″. Preferably, metal plate 93″ is insert molded onto a plastic base material. The metal portion contacts dosing plate 180 in the assembled device, providing a very flat, smooth and rigid surface to prevent powder leakage from the reservoir. In addition, the metal dissipates any static electricity charges generated by friction between surfaces during dose loading operations, which charges can adversely affect powder flow into and out of the dosing station.

As shown in FIGS. 10-14, driving body 120 includes a circular top wall 122 having an annular skirt 124 extending downwardly from the periphery of circular top wall 122.

Annular skirt 124 includes an upper annular skirt section 126 with its upper end extending downwardly from the periphery of circular top wall 122, and a lower annular skirt section 128 extending downwardly from the lower end of upper annular skirt section 126. Lower annular skirt section 128 has an inner and outer diameter greater than the inner and outer diameters, respectively, of upper annular skirt section 126. Accordingly, an inner annular shoulder 130 is formed at the lower edge of upper annular skirt section 126, along the inside of annular skirt 124. However, the outer surface of the transition area between upper annular skirt section 126 and lower annular skirt section 128 is formed as a frusto-conical surface 132.

Further, the inner diameter of lower annular skirt section 128 is substantially the same as the outer diameter of upper annular skirt section 28 of reservoir body 22 and the inner diameter of upper annular skirt section 126 is substantially the same as the outer diameter of peripheral securing wall 74 of reservoir body 22. Accordingly, reservoir body 22 fits into driving body 120 with a close fit.

In order to lock reservoir body 22 and driving body 120 together in such position, an annular holding area 138, such as in the form of a channel, is defined on the inner surface of upper skirt section 126 formed parallel to and spaced above annular shoulder 130. Thus, when reservoir body 22 is inserted within driving body 120 in the manner described above, lip 80 at the upper end of peripheral securing wall 74, due to the resilience of the plastic pieces, rides along the inner surface of upper skirt portion 126 and is held within annular holding area 138, as shown in FIG. 15. Ribs or other protrusions may be defined adjacent to the holding area 138 to enhance the holding effect thereof.

Circular top wall 122 is formed with two circular openings 142, 142′ which are aligned with to receive venturi conduits 64, 64′ so that the upper edges of venturi conduits 64, 64′ are substantially flush with the upper surface of circular top wall 122 (FIG. 15).

Two circular plug conduits 144, 144′ depend downwardly from the lower surface of circular top wall 122 which are in alignment with powder supply conduits 60, 60′, respectively. Circular plug conduits 144, 144′ have each an outer diameter substantially equal to or slightly greater than the, inside diameter of the corresponding powder supply conduit 60, 60′. Thus, the plug conduits 144, 144′ close the upper open ends of powder supply conduits 60, 60′ when reservoir body 22 is assembled with driving body 120. Therefore, powder 62, 62′ can only escape through manifolds 46, 46′; openings, 55, 55′; and substantially oval holes 98, 98′.

Further, curved retaining walls 148, 148′ extend downwardly from the lower surface of circular top wall 122 in partial surrounding relation to circular openings 142, 142′, respectively, to ensure further separation between powder supply conduits 60, 60′ and frusto-conical venturi conduits 64, 64′ when reservoir body 22 and driving body 120 are assembled.

In order to provide for secondary air flow, as will be described hereinafter, the wall defining upper annular skirt section 126 extends inwardly in the radial direction to form a first outer air passage 150 adjacent to circular opening 142′ in the circumferential direction of driving body 120 and a second outer air passage 152 adjacent to circular opening 142.

Short, axially extending upper guide walls 154 and 156 are formed along a common circular arc spaced slightly inwardly from the periphery on the upper surface of circular top wall 122 in order to secure a nozzle to driving body 120, as will be described in greater detail hereinafter. Specifically, upper guide wall 154 is formed circumferentially along the larger arc between air passages 150 and 152; and upper guide wall 156 is formed circumferentially along the smaller arc between air passages 150 and 152. The common circular arc along which upper guide walls 154 and 156 extend is spaced slightly from the peripheral edge of circular top wall 122 so as to define an annular retaining ledge 159 on circular top wall 122, positioned outwardly of upper guide walls 154 and 156 in the radial direction.

Four substantially equiangularly arranged, elongated arcuate recesses 158a-158d are formed on retaining ledge 159, the purpose for which will be apparent from the discussion hereinafter. Recesses 158a-158d extend along different arcuate distances. For example, recesses 158a, 158b and 158c may extend for arcuate distances of 45 degrees; and recess 158d may extend for an arcuate distance of 28 degrees.

Further, lower annular skirt section 128 is cut away at spaced-apart positions thereof to form driving openings 164, 166, 169 containing spring fingers 161, 163, 165, respectively, extending downwardly and slightly outwardly from their respective connections 167 at the intersection of upper annular skirt section 126 and lower annular skirt section 128. Spring fingers 161, 163, 165, as shown, extend below the lower edge of lower annular skirt section 128. As will be described hereinafter, driving openings 164, 166, 169 are engaged to rotate driving body 120. As shown, each spring finger 161, 163, 165 is bent or formed into a concave shape so as to have a depression 171 therein, substantially centrally located with respect to the lengthwise direction thereof.

In order to provide metered doses of powder 62, 62′ from respective powder supply conduits 60, 60′ to venturi conduits 64, 64′, a metering dose plate 180 is positioned within upper annular skirt section 28 of reservoir body 22, immediately below reservoir plug 90. As shown in FIGS. 16-19, metering dose plate 180 includes a thin disc 182 having two dose holes 184, 184′ near the periphery thereof which function as powder receptacles, that is, for holding metered doses of powder 62, 62′. It is preferred that the dose holes 184, 184′ be at diametrically opposite locations. In order to prevent the metered dose of powder from falling through dose holes 184, 184′, powder retainers 186, 186′ are formed in covering relation to the lower surface of disc 182, extending at least over dose holes 184, 184′. Preferably, powder retainers 186, 186′ are formed by a mesh screen, filter, porous material or the like which has a minimal restrictive effect on gas flow therethrough, while preventing appreciable loss of powdered medicament below the lower surface of disc 182. Powder retainers 186, 186′ can be fabricated from any suitable material, including cellulosics, polymerics, metals, ceramics, glasses or composites thereof, exemplary useful materials including sintered porous plastics, porous polymer membranes, natural or synthetic woven fabrics, nonwoven synthetic fabrics and the like. More specifically, useful materials include polyester and polyolefin woven mesh, and porous membranes of polyolefins, polycarbonates, poly-tetrafluoroethylene, polyvinylidene dichloride, and mixed esters of cellulose.

Powder retainers 186, 186′ may be configured and affixed to metering dose plate 180 in any known manner. By way of non-limiting example, powder retainers 186, 186′ may be affixed in recesses formed in metering dose plate 180 as described in U.S. Pat. No. 6,240,918.

In accordance with the present invention, to easily and accurately form powder retainers 186, 186′ within corresponding recesses, metering dose plate 180 is preferably formed by an insert molding operation. The insert molding operation described in U.S. Pat. No. 6,240,918 for forming a metering dose plate may be utilized. In addition, U.S. Pat. No. 6,240,918 describes an alternative manner of configuring and affixing powder retainers 186, 186′ to metering dose plate which also may be utilized.

An annular mounting post 188 extends downwardly from the lower surface of disc 182 and is centrally located thereon. Annular mounting post 188 is formed with a bar 190 extending axially along the inner surface of mounting post 188 in axial relation to metered dose holes 184, 184′. Bar 190 extends from the lower surface of disc 182 to a position slightly spaced from the lower edge of mounting post 188, and preferably has a square cross-sectional configuration. As will be understood from the description hereinafter, bar 190 ensures that metering dose plate 180 will remain stationary with respect to powder housing 20 when powder housing 20, which includes reservoir body 22, reservoir plug 90 and driving body 120, is rotated.

In operation, metered dose hole 184 is initially in alignment with frusto-conical venturi conduit 64 and metered dose hole 184′ is initially in alignment with frusto-conical venturi conduit 64′. As will be explained hereinafter, powder housing 20 is only preferably permitted to rotate an angle of 120 degrees relative to metering dose plate 180. As will be appreciated by those skilled in the art, other working angular ranges are useable consistent with the subject invention. During initial priming rotation, metered dose holes 184, 184′ pass under manifolds 46, 46′ and substantially oval holes 98, 98′. As a result, powder 62, 62′ falls respectively within and is scraped into metered dose holes 184, 184′. Specifically, the side walls defining substantially oval holes 98, 98′ function to scrape the powder 62, 62′ into metered dose holes 184, 184′. It will be appreciated that, since oval holes 98, 98′ are spaced less than the range of motion of the powder housing 20 relative to metering dose plate 180 from circular holes 94, 94′ metered dose hole 184 travels completely past oval hole 98 and manifold 46, while metered dose hole 184′ travels past oval hole 98 and manifold 46′ during an actuation of the device 10. Then, during the return rotation back to the initial position, metered dose hole 184 passes back under manifold 46 and substantially oval holes 98′ into alignment with venturi conduit 64, while metered dose hole 184′ passes back under manifold 46′ and substantially oval hole 98 into alignment with venturi conduit 64′. During this return travel, the side walls defining substantially oval holes 98, 98′ again function to scrape the powder 62, 62′ into metered dose holes 184, 184′ thus ensuring that metered dose holes 184, 184′ are completely and accurately filled. Thus, the scraping action is provided during both counterclockwise and clockwise rotation, that is, both during the rotating loading stage and the reverse movement to the inhalation stage. When metered dose holes 184, 184′ are aligned with venturi conduits 64, 64′, respectively, it is then only necessary for the user to inhale through venturi conduit 64, 64′, causing a draw and suction through metered dose holes 184, 184′ wherein the metered doses of powder 62, 62′ are drawn up through venturi conduits 64, 64′ and delivered to the user.

As will be understood by those skilled in the art, relative movement between the supply conduits 60, 60′ and the metering dose plate is required for actuation of the dispenser 10 (both priming and dispensing doses). The relative movement is described and shown as being rotating movement. It is to be understood that linear relative movement may be also utilized with the components be correspondingly formed (i.e., the radius of rotation is set to infinity (∞)).

With reference to FIG. 16, metered dose holes 184, 184′, are shown to be of the same size. Thus, the metered dose boles 184, 184′ are configured to provide the same size doses. As shown in FIGS. 17 and 21, the metered dose holes 184, 184′ may be formed of different dimensions. In this manner, the metered dose holes 184, 184′ may accommodate different size doses.

Regardless of the size of the metered dose holes 184, 184′, the powders 62, 62′ may be of different types. The powders 62, 62′ may be of different drugs (e.g., different composition; same composition, different strength) and/or may be provided with different physical properties or characteristics (e.g., have different aerodynamic particle size distribution (APSD) so as to reach different target areas in a patient's respiratory system). In addition, the powders 62, 62′ can be introduced into the discharge stream at different points thereby allowing the powders 62, 62′ to be subjected to different discharge conditions (e.g., greater or less discharge velocity; subjected to more or less deagglomeration). One type of powder may be administered in greater amount than the other powder in forming a combination. As such, different sized doses of the powders 62, 62′ may be combined. Also, different amounts of the powders 62, 62′ may be initially provided in the powder supply conduits 60, 60′, respectively. With this arrangement, dosing of different drugs may be conducted over different durations. For example, one of the powders 62 may be provided for a seven-day administration, while the other of the powders 62′ may be provided for a longer duration administration (e.g., 7-30 days). By way of non-limiting example, an antibiotic may be provided for a relatively short-term (e.g., seven days) with a steroid being provided over a longer term (e.g., twenty-one days). With this arrangement, the dispenser 10 would dispense both drugs for the first term and then only the longer-duration drug. The dispenser 10 allows incompatible (e.g., chemically incompatible) drugs to be simultaneously stored and delivered.

Suitable at least one active pharmaceutical agents useable with the subject invention include but are not limited to an anticholinergic, a corticosteroid, a long acting beta agonist, short acting beta agonist, a phosphodiesterase IV inhibitor. Suitable medicaments may be useful for the prevention or treatment of a respiratory, inflammatory or obstructive airway disease. Examples of such diseases include asthma or chronic obstructive pulmonary disease.

Suitable anticholinergics include (R)-3-[2-hydroxy-2,2-(dithien-2-yl)acetoxy]-1-1[2-(phenyl)ethyl]-1-azoniabicyclo[2.2.2]octane, glycopyrrolate, ipratropium bromide, oxitropium bromide, atropine methyl nitrate, atropine sulfate, ipratropium, belladonna extract, scopolamine, scopolamine methobromide, methscopolamine, homatropine methobromide, hyoscyamine, isopriopramide, orphenadrine, benzalkonium chloride, tiotropium bromide, GSK202405, an individual isomer of any of the above or a pharmaceutically acceptable salt or hydrate of any of the above, or a combination of two or more of the above.

Suitable corticosteroids includes mometasone furoate; beclomethasone dipropionate; budesonide; fluticasone; dexamethasone; flunisolide; triamcinolone; (22R)-6.alpha., 9.alpha.-difluoro-11.beta., 21-dihydroxy-16.alpha., 17.alpha.-propylmethylenedioxy-4-pregnen-3,20-dione, tipredane, GSK685698, GSK799943 or a pharmaceutically acceptable salt or hydrate of any of the above, or a combination of two or more of the above.

Suitable long acting beta agonist include carmoterol, indacaterol, TA-2005, salmeterol, formoterol, or a pharmaceutically acceptable salt or hydrate of any of the above, or a combination of two or more of the above. Suitable short acting beta agonist include albuterol, terbutaline sulfate, bitolterol mesylate, levalbuterol, metaproterenol sulfate, pirbuterol acetate or a pharmaceutically acceptable salt or hydrate of any of the above, or a combination of two or more of the above.

Suitable phosphodiesterase IV inhibitors include cilomilast, roflumilast, tetomilast, 1-[[5-(1(S)-aminoethyl)-2-[8-methoxy-2-(trifluoromethyl)-5-quinolinyl]-4-oxazolyl]carbonyl]-4(R)-[(cyclopropylcarbonyl)amino]-L-proline, ethyl ester or a pharmaceutically acceptable salt or hydrate of any of the above, or a combination of two or more of the above.

In certain embodiments of the present invention the at least one active pharmaceutical agent includes a corticosteroid, such as mometasone furoate. Mometasone furoate is an anti-inflammatory corticosteroid having the chemical name, 9,21-Dichloro-11(beta), 17-dihydroxy-16(alpha)-methylpregna-1,4-diene-3,20-dione 17-(2 furoate). It is practically insoluble in water; slightly soluble in methanol, ethanol, and isopropanol; soluble in acetone and chloroform; and freely soluble in tetrahydrofuran. Its partition coefficient between octanol and water is greater than 5000. Mometasone can exist in various hydrated, crystalline and enantiomeric forms, e.g., as a monohydrate.

The at least one APA may be in the form of an agglomerate. Agglomerates of drug alone or with another substance may be utilized, such as those agglomerates described in U.S. Pat. No. 6,503,537, which is incorporated herein. Any method of agglomerating the solid binder and the pharmacologically active agent may be used. Useful agglomerating methods include those which can be accomplished without converting the amorphous content of the solid binder to a crystalline form, prematurely, and which does not require the use of additional binder, can be practiced in accordance with the present invention.

Useful agglomerates include agglomerates ranging in size from between about 100 to about 1500 μm. The agglomerates may have an average size of between about 300 and about 1,000 μm. Useful agglomerates may have a bulk density which ranges from between about 0.2 to about 0.4 g/cm³ or between about 0.29 to about 0.38 g/cm³.

It is useful to have a tight particle size distribution. In this context, particle size refers to the size of the agglomerates. Preferably, no more than about 10% of the agglomerates are 50% smaller or 50% larger than the mean or target agglomerate size. For example, for an agglomerate of 300 μm, no more than about 10% of the agglomerates will be smaller than about 150 μm or larger than about 450 μm.

A useful method of preparing the agglomerates in accordance with the invention which meets all of the foregoing criteria involves mixing preselected amounts of one or more pharmacologically active agent(s) and the micronized, amorphous content containing, dry solid binder in a ratio of between about 100:1 and about 1:500; between about 100:1 and about 1:300 (drug:binder); between about 20:1 to about 1:20 or a ratio of about 1:3 to about 1:10 relative to the amount of the solid binder.

Useful agglomerates may have a strength which ranges from between about 50 mg and about 5,000 mg and most preferably between about 200 mg and about 1,500 mg. The crush strength was tested on a Seiko TMA/SS 120C Thermomechanical Analyzer available from Seiko Instruments, Inc. Tokyo, Japan, using procedures available from the manufacturer. It should be noted that strength measured in this manner is influenced by the quality and extent of the interparticulate crystalline bonding described herein. However, the size of the agglomerates also plays a role in the measured crush strength. Generally, larger agglomerates require more force to crush than do the smaller particles.

In order to provide for relative rotation, metering dose plate 180 is non-rotatably mounted on, and powder housing 20 is rotatably mounted on, a base 200, shown in FIGS. 3, 4 and 22-26. Base 200 includes a circular top wall 202 having an annular skirt 204 extending downwardly from the periphery thereof. The peripheral edge of circular top wall 202 is cut-away to define an outer annular ledge 206. An annular supporting lip 208 is formed on the outer surface of annular skirt 204 at the lower end thereof, so as to extend outwardly therefrom in the radial direction of annular skirt 204. An annular wall 209 having a diameter less than that of supporting lip 208 is formed at the upper end of supporting lip 208. As shown in FIG. 4, annular wall 209 can have a plurality of axially spaced apart, annular teeth 211 on the outer surface thereof. In addition, an annular retaining rim 210 is formed on the upper, outer surface of annular skirt 204, parallel to supporting lip 208 and annular wall 209, and spaced above annular wall 209, so as to extend outwardly from annular skirt 204 in the radial direction thereof. Retaining rim 210 has a diameter slightly less than the diameter of annular wall 209. Thus, an annular retaining gap 212 is formed between annular wall 209 and retaining rim 210.

Further, a small post 214 is formed, extending upwardly from annular wall 209 to a height above retaining rim 210, but below top wall 202. Post 214 has an outside diameter equal to that of annular wall 209, and also is connected with retaining rim 210 and extends within gap 212.

A cylindrical boss 216 is formed centrally and axially on the upper surface of circular top wall 202, with an upper annular portion 217 thereof partially cut-away and a radial segment 219 thereof also cut away. A coaxial retaining post 218 of lesser diameter than cylindrical boss 216 is formed at the upper end of cylindrical boss 216. Accordingly, an outer annular ledge 220 is formed at the upper edge of cylindrical boss 216. Retaining post 218 has an outer diameter slightly less than the inner diameter of annular mounting post 188 of metering dose plate 180. Retaining post 218 is formed with a slot 222 along the length thereof. Accordingly, due to bar 190 and slot 222, mounting post 188 of metering dose plate 180 is retained on retaining post 218 in a non-rotatable manner to ensure that metering dose plate 180 will remain stationary with respect to powder housing 20 when powder housing 20, which includes reservoir body 22, reservoir plug 90 and driving body 120, is rotated.

Two short stub walls 221 and 223 are formed on the upper surface of top wall 202, immediately on opposite sides of cylindrical boss 216. Stub walls 221 and 223 are angled with respect to each other at an angle of approximately 30 degrees.

As part of a counter mechanism which will be described in greater detail hereinafter, a first rotation prevention spring detent 224 is mounted in a cantilever manner on circular top wall 202. Specifically, a curved vertical detent supporting wall 226 extends upwardly from circular top wall 202 at a position substantially midway between annular ledge 206 and cylindrical boss 216, and first rotation prevention spring detent 224 extends from one edge 228 of detent supporting wall 226, parallel to and spaced above circular top wall 202. Further, the free end of first rotation prevention spring detent 224 is provided with an outward radially directed tab 230 thereat.

Also as part of the counter mechanism which will be described in greater detail hereinafter, a second rotation prevention spring detent 232 is mounted in a cantilever manner on circular top wall 202. Specifically, second rotation prevention spring detent 232 extends from edge 228 of detent supporting wall 226, parallel to and spaced above circular top wall 202 and parallel to and spaced above first rotation prevention spring detent 224. The free end of second rotation prevention spring detent 232 is provided with an outward radially directed tab 234.

A triangular shaped sectored recess 236 is formed in circular top wall 202 in correspondence with detents 224 and 232, and diametrically opposite to post 214. Specifically, recess 236 includes a first radial boundary 240 substantially in line with the connected end of detent 232, and a second boundary 242 extending in alignment with the lengthwise direction of detent 232.

Further, a shallow recess 243 is provided at the outer radial edge of annular ledge 206, in alignment with sectored recess 236, and diametrically opposite post 214.

In order to spring bias metering dose plate 180 into engagement with the lower surface of thin circular plate 92 of reservoir plug 90 and to ensure that powder 62, 62′ can only be inhaled when metered dose holes 184, 184′ are in alignment with venturi conduits 64, 64′, a biasing assembly is provided.

The biasing assembly includes a lower spring retainer 260 mounted on annular ledge 220, over retaining post 218, as shown in FIGS. 3, 4 and 27-31. Specifically, lower spring retainer 260 includes a disc 262 having a central opening 264 sized to receive retaining post 218. An annular boss 266 extends from the lower surface of disc 262 in surrounding relation to central opening 264. When retaining post 218 extends through annular boss 266 and central opening 264, the lower edge of annular boss 266 seats upon annular ledge 220.

An upper annular retaining lip 268 extends upwardly from the peripheral edge of disc 262. Further, radially extending driven ears 270, 271 and 272 are formed along the peripheral edge of annular lip 268. Ear 270 has a width substantially equal to the width of drive slot 36 of reservoir body 22 so as to fit therein and be driven thereby, and ears 271, 272 have widths substantially equal to the widths of drive slots 34, 35, respectively of reservoir body 22 so as to fit therein and be driven thereby.

Further, an arcuate pawl driving wall 274 extends from the lower surface of disc 262 between annular boss 266 and the periphery of disc 262. Pawl driving wall 274 includes opposite pawl driving ends 276 and 278, as will be described in greater detail hereinafter with reference to the counter mechanism.

The biasing assembly further includes a coil spring 290 having one end seated on the upper surface of disc 262 of lower spring retainer 260, and restrained thereon by annular retaining lip 268.

As shown in FIGS. 3, 4 and 32-33, the biasing assembly further includes a support plate 300 which supports metering dose plate 180, functions as an upper spring retainer, biases metering dose plate 180 against the lower surface of thin circular plate 92 of reservoir plug 90, and permits suction through metered dose holes 184, 184′ only when metered dose holes 184, 184′ are in alignment with venturi conduits 64, 64′.

Specifically, support plate 300 is formed by a disc 302 having an annular retaining lip 304 extending downwardly from the peripheral edge of disc 302.

Three radially extending driven ears 306, 307, 308 are formed on the peripheral edge of annular lip 304. Ear 306 has a width substantially equal to the width of drive slot 36 of reservoir body 22 so as to fit therein and be driven thereby, and ears 307 and 308 have widths substantially equal to the widths of drive slots 34 and 35 of reservoir body 22 so as to fit therein and be driven thereby. The heights of ears 306, 307, 308 are less than the height of annular lip 304, and lower surfaces of ears 306, 307, 308 are substantially flush with the lower edge of annular lip 304, although the invention is not so limited.

In addition, a central circular hole 310 is formed in disc 302 and is sized to rotatably receive annular mounting post 188 of metering dose plate 180 therein. A radially extending slot 312 extends from and is in communication with circular hole 310. Slot 312 extends outwardly in the radial direction by a distance such that the radially outer part of slot 312 overlaps metered dose holes 184, 184′ when metered dose holes 184, 184′ are in alignment with venturi conduits 64, 64′ and is out of alignment with, and thereby does not overlap, metered dose holes 184, 184′ at all other times.

As described above, powder retainers 186, 186′ are formed by a mesh screen, filter, porous material or the like which has a minimal restrictive effect on gas flow therethrough. However, when a mesh screen or the like is used, there is a reduction in gas flow, and thereby of any suction by the user, of approximately 35%. According to an alternative embodiment, as shown in FIG. 34, powder retainer 186 comprised of a mesh screen or the like can be relocated to the lower surface of disc 302 of support plate 300, under slot 312. Therefore, although the mesh screen or the like reduces the gas flow through radially extending slot 312, this does not effectively restrict the gas flow through metered dose hole 184 or 184′ which is smaller than slot 312. Thus, primary air flow is independent of the cross-sectional width of metering dose plate 180. Further, there is no mesh powder retainer 186 at metered dose hole 184 or 184′ to reduce air flow through metered dose holes 184, 184′.

It will be appreciated from the above description that metering dose plate 180 is held stationary on base 200, due to bar 190 and slot 222. Further, powder housing 20, comprised of reservoir body 22, reservoir plug 100 and driving body 120, is rotatably mounted with respect to base 200 and metering dose plate 180.

In addition, support plate 300 is biased into engagement with the lower surface of metering dose plate 180 so as to support the same. In the operation, radially extending slot 312 is in alignment with metered dose holes 184, 184′ only when metered dose holes 184, 184′ are in alignment with venturi conduits 64, 64′. Thus, any powder 62, 62′ within metered dose holes 184, 184′ when metered dose holes 184, 184′ are out of alignment with venturi conduits 64, 64′ are sandwiched in metered dose holes 184, 184′ by mesh powder retainers 186, 186′ and the upper surface of disc 302 of support plate 300 at its lower end, and by the lower surface of thin circular plate 92 of reservoir plug 90 at its upper end. As will be discussed in greater detail hereinafter, in the stored or inactive position of metered powder dose dispenser 10, metered dose holes 184, 184′ are primed, and are positioned out of alignment with radially extending slot 312. In such position, powder 62, 62′ within metered dose holes 184, 184′ is held between the upper surface of disc 302 of support plate 300 and the lower surface of thin circular plate 92 of reservoir plug 90, and therefore cannot escape metered dose holes 184, 184′.

In order to positively hold all of the above elements together, metered powder dose dispenser 10 further includes an adapter 320, as shown in FIGS. 3, 4 and 35-36. As shown therein, adapter 320 includes a lower annular wall 322 having an inner diameter larger than the outer diameter of lower annular skirt section 30 of reservoir body 22 so as to easily fit thereover. The inner diameter of lower annular wall 322 is also slightly larger than the outer diameter of annular skirt 204 of base 200 so as to fit thereover, but slightly less than the outer diameter of annular retaining rim 210 of base 200.

An annular groove 324 is formed at the inner, lower end of lower annular wall 322, slightly spaced above the lower edge thereof. Accordingly, due to the resilience of the plastic pieces, when adapter 320 is inserted over base 200 and pushed down thereon, retaining rim 210 of base 200 snaps into annular groove 324 to hold adapter 320 on base 200. At such time, annular teeth 211 can engage the inner surface of lower annular wall 322, as shown in FIG. 4.

In order to obtain and maintain correct alignment between adapter 320 and base 200, adapter 320 is provided with a small slot 326 within groove 324. Slot 326 has a width substantially equal to that of small post 214 in base 200 so as to receive the same therein. Of course, it will be appreciated that post 214 can be provided in adapter 320 and slot 326 can be provided in base 200, that is, with a reversal of parts. Thus, rotation of adapter 320 causes base 200 to rotate therewith.

The outer surface of lower annular wall 322 is preferably provided with a gripping surface 328 formed by undulations, knurling or the like, to enhance the gripping and rotation of metered powder dose dispenser 10.

A rectangular opening 329 is formed in lower annular wall 322, substantially diametrically opposite to slot 326, and substantially centrally along the height of lower annular wall 322. A rectangular transparent plastic window 330 is fixed in opening 329 by an adhesive, welding or the like. Window 330 is used with the counter mechanism which will be described in greater detail hereinafter.

Adapter 320 further includes an upper annular wall 332 of a lesser diameter than lower annular wall 322, and connected to the upper end of lower annular wall 322 by an outer annular shoulder 334.

An annular biasing lip 338 is formed on the inner surface of upper annular wall 332. When adapter 320 is pushed down so as to lock adapter 320 onto base 200, as described above, annular biasing lip 338 seats on outer annular shoulder 32 of reservoir body 22, and thereby biases reservoir body 22 down against the force of coil spring 290. Accordingly, coil spring 290 is compressed so that a biasing force always forces support plate 300 into abutment with metering dose plate 180, and always forces metering dose plate 180 into abutment with reservoir plug 90. However, such biasing action still permits rotation of reservoir body 22 relative to adapter 320 and metering dose plate 180.

At the same time, this compression ensures that driven ears 270 and 306 will always be located within drive slot 36 and driven ears 271, 272 and 307, 308 will always be located within drive slots 34 and 35, so that rotation of reservoir body 22 will cause consequent rotation of lower spring retainer 260 and support plate 300. Because metering dose plate 180 is held stationary on base 200, due to bar 190 and slot 222, powder housing 20 (comprised of reservoir body 22, reservoir plug 90 and driving body 120), lower spring retainer 260 and support plate 300, are rotatably mounted with respect to base 200, metering dose plate 180 and adapter 320.

In the assembled condition discussed above, the lower edge of lower annular skirt section 128 of driving body 120 rests and rotates on the upper edge of upper annular wall 332 of adapter 320. In order to provide air flow through metered dose holes 184, 184′ of metering dose plate 180, spaced-apart recesses 340, 341, 342 are formed in upper annular wall 332, extending from the upper edge of upper annular wall to annular biasing lip 338. Recess 340 has a width identical to the width of drive slot 36, while recesses 341, 342 have widths identical to the widths of drive slots 34, 35. When metered dose holes 184, 184′ are aligned with venturi conduits 64, 64′ of reservoir body 22 and with radially extending slot 312 of support plate 300, recess 340 is in alignment with drive slot 36 and recesses 341, 342 are in alignment with drive slots 34, 35. Accordingly, suction on venturi conduits 64, 64′ causes air to flow through recess 340 and drive slot 36 and through recesses 341, 342 and drive slots 34, 35, and then through radially extending slot 312, metered dose holes 184, 184′ and venturi conduits 64, 64′ to deliver the metered doses of powder 62, 62′ in metered dose holes 184, 184′ to a user of dispenser 10.

In addition, the recesses 340, 341, 342 are oriented so as to receive spring fingers 161, 163, 165 to lock the assembly in position after the cap has been removed, as discussed below.

As shown in FIGS. 35-36, recesses 340, 341, 342, each have one side thereof with a bevel 345 toward the inside surface thereof, the purpose for which will become apparent hereinafter.

A double helical cam track 352 is formed on the outer surface of upper annular wall 332, the purpose for which will become apparent from the description which follows. As is apparent, the walls 353 that form double helical track 352 have a substantially square cross-section, the purpose for which will become apparent from the discussion hereinafter with respect to the cap. Further, the entry 351 to each cam track 352 is formed as a vertical drop zone before rotation can begin, thus ensuring accurate registry of the closure cap and thereby, accurate operation of dispenser 10.

In order to ensure that the powder is de-agglomerated and properly mixed with the suction air from the open upper end of upper venturi conduit section 68 of venturi conduit 64, a swirl nozzle 380, as shown in FIGS. 37-39, is mounted to the upper end of reservoir body 22. Air which contains agglomerated powder particles flows from upper venturi conduit section 68 into the swirl nozzle. Mechanical de-agglomeration is an important function of the swirl nozzle. In addition, swirl nozzle 380 acts as a mixing chamber or mixing the powders 62, 62′ together. The powders 62, 62′ may be delivered separately by venturi conduits 64, 64′, but then mixed in the swirl nozzle 380 for delivery as a single dose.

As will be appreciated by those skilled in the art, various swirl nozzle configurations are usable with the subject invention. By way of non-limiting example, swirl nozzle 380 includes a circular top wall 382 and an annular side wall 384 extending downwardly from the periphery of top wall 382. Annular side wall 384 has an outer diameter substantially equal to the outer diameter of upper annular skirt section 126 of driving body 120. Further, the inner connecting region 386 between circular top wall 382 and annular side wall 384 is curved to provide a smooth transition therebetween and thereby to provide a smooth flow path for powder 62, 62′. In other words, the inner area defined by circular top wall 382, annular side wall 384 and inner connecting region 386 has a somewhat partial toroidal configuration. The outer connecting region 390 therebetween, however, forms a substantially right angle in cross-section between circular top wall 382 and annular side wall 384.

In order to secure swirl nozzle 380 onto the upper end of driving body 120, and particularly, onto annular retaining ledge 159 of driving body 120, four spiked ribs 392, 393, 394 and 396 are equiangularly formed extending down from the lower edge of annular side wall 384. Spiked ribs 392, 393, 394 and 396 extend arcuate distances which are different from each other and which correspond identically with the arcuate distances of arcuate recesses 158a-158d, respectively, of driving body 120 so that swirl nozzle 380 is assembled at a predetermined position with driving body 120. For example, spiked ribs 392 and 394 can extend for an arcuate distance of 40 degrees; spiked rib 393 for an arcuate distance of 23 degrees; and spiked rib 396 for an arcuate distance of 40 degrees. Spiked ribs 392, 393, 394 and 396 extend along a common circle having a diameter equal to the common circle around which recesses 158a-158d extend. Thus, spiked ribs 392, 393, 394 and 396 extend within recesses 158a-158d, respectively, with a two degree adjustment clearance. Preferably, each spiked rib 392, 393, 394 and 396 has a tapered end with a substantially triangular cross-sectional configuration.

During an inhalation process, swirl nozzle 380 and the mouthpiece (discussed later) secured thereto might detach from driving body 120 and be swallowed. Therefore, in order to fixedly secure swirl nozzle 380 onto driving body 120, an ultrasonic welding operation is performed. Specifically, ultrasonic energy is directed toward spiked ribs 392, 393, 394 and 396. In such case, the spiked or sharp ends of ribs 392, 393, 394 and 396 function as energy directors which absorb greater amounts of energy. As a result, the plastic material of spiked ribs 392, 393, 394 and 396 is fused into the plastic material of recesses 158a-158d to secure swirl nozzle 380 on driving body 120. With this arrangement, there is a uniform energy that is applied for securing swirl nozzle 380, and an automatic operation can be used to perform such securing operation, achieving a consistency at all times.

It will be appreciated that, in such position, first and second outer air passages 150 and 152 extend inwardly of annular side wall 384 to supply secondary air flow thereto which mixes with the air/powder mixture from venturi conduits 64, 64′ which is also supplied to the interior of annular side wall 384.

Circular top wall 382 has a central opening 402, and a supply chimney 404 is formed on the upper surface of circular top wall 384 in surrounding relation to central opening 402.

In order to break up the powder agglomerates, prior to supplying the same through supply chimney 404, a curved spiral-like wall 406 extends downwardly from circular top wall 382 and is connected at one end 408 to annular side wall 384. Specifically, curved wall 406 extends in a curvilinear manner from end 408, and partially about central opening 402 to an opposite end 410. Thus, a gap 409 is provided between end 410 and the remainder of curved wall 406. The height of curved wall 406 is equal to that of annular side wall 384 so that the lower edge of curved wall 406 sits on circular top wall 122 of driving body 120 when swirl nozzle 380 is assembled with driving body 120, as described above. Curved wall 406 is effectively formed in two sections, namely, a first section starting from end 410 and extending partially about central opening 402, for example, for 165-227 degrees, and a second section extending from the end of the first section to end 408 along a larger radius than the first section.

As will be appreciated, curved wall 406 defines a swirl cavity 412, such that the powder from venturi conduits 64, 64′ enters swirl cavity 412 and continuously changes direction as it increases in velocity, prior to entering supply chimney 404. Thus, the powder agglomerates constantly impact against circular top wall 382, annular side wall 384 and curved wall 406 within swirl cavity 412. Further, the agglomerates collide with each other which results in a mutual grinding or shattering action between the agglomerates. At the same time, secondary air flow from first and second outer air passages 150 and 152 enters swirl cavity 412, respectively, to accelerate movement of the powder agglomerates in swirl cavity 412. The constant impacts of the powder agglomerates on the walls defining swirl cavity 412 cause the agglomerates to break up into micronized powder upon impact. Basically, as long as the powder agglomerates travel with sufficient velocity, there will be sufficient kinetic energy to break up the agglomerates.

Further, curved wall 406 and, particularly, swirl cavity 412, first changes the direction of powder 62 from an axial direction of venturi conduits 64, 64′ to a transverse direction substantially perpendicular to the axial direction. In this transverse direction, powder 62, 62′ is then forced to continuously change direction in the transverse direction of swirl cavity 412. Upon exiting swirl cavity 412, the direction of powder 62, 62′ is again changed to an axial direction through supply chimney 404, while retaining a swirl component of the flow, that is, while swirling spirally through chimney 404. Since the micronized powder and any remaining agglomerates maintain the swirl imparted thereto from swirl cavity 412, the swirling flow applies a centrifugal force to the micronized powder and remaining agglomerates, creating additional impacts in supply chimney 404 so as to result in further breaking up of the remaining agglomerates.

Most of the agglomerate break-up should take place, however, in swirl cavity 412. The velocity attained by an agglomerate depends on the drag or suction force, the inertia of the agglomerate, and the length of swirl cavity 412, that is, the time the drag force acts on the agglomerate. Because of its inertia, the agglomerate impacts a wall in swirl cavity 412 to convert the same to micronized powder.

In addition, with the present invention, chimney 404 is provided with vertically oriented grooves or flutes 405 extending along the inner wall thereof. Flutes 405 provide more surfaces against which the agglomerates can impact against. Flutes 405 are shown as being formed by six vertical concave wall sections 411 of a first radius, which are interconnected by six vertical concave wall sections 413 of a larger radius, or even of a flat, planar configuration, that is, infinite radius. However, any other suitable arrangement can be provided. It is preferable, however, that whatever arrangement is provided, flutes 405 or any other configuration are vertically oriented, and thereby provide an irregular vertically oriented surface. Further, as shown, flutes 405 preferably extend from the upper edge of chimney 404 to the upper edge of curved wall 406, although the present invention is not so limited.

Flutes 405 aid in the break-up of agglomerates that require greater de-agglomeration forces to disperse.

Experiments have shown that fluted swirl nozzle 380 increases the respirable fraction over a similar swirl nozzle which is not fluted. Specifically, for hard agglomerates, such as those having a bulk density in the range of 0.29-0.36 g/ml, the same swirl nozzle without flutes provided approximately a 10% respirable fraction, while a fluted swirl nozzle provided approximately a 35% respirable fraction. “Respirable fraction” for purposes of these experiments is the percentage of total particles delivered from the nozzle that are less than or equal to 6.8 micrometers in diameter, as determined using a multi-stage liquid impinger. In the experiments, the formulation was mometasone and lactose agglomerates in a component weight ratio of 1 to 5.8.

In addition to breaking up agglomerates, swirl nozzle 380 must meet additional constraints. For example, the pressure drop through the powder inhaler should desirably be lower than about 20 inches of a water column (5 Kpa) for ease of use by persons with impaired respiratory function, yet sufficiently high to permit significant primary air flow through metered dose holes 184, 184′. The pressure drop through swirl nozzle 380 can be changed by varying the angle between end 410 and the position where the first and second sections of curved wall 406 meet, that is, where the second section leaves central opening 402. In a presently preferred embodiment, this angle is about 165°, although this value may change depending upon the required pressure drop.

Further, an annular mouthpiece securing wall 418 is formed on the upper surface of circular top wall 382, spaced slightly inwardly from the peripheral edge thereof. As a result, an annular ledge 420 is formed on the upper surface of circular top wall 382, outwardly of annular mouthpiece securing wall 418. Further, an annular lip 422 extends outwardly in the radial direction from the upper end of annular mouthpiece securing wall 418.

Also, gear teeth 424 are provided on the upper edge of annular mouthpiece securing wall 418. Although forty gear teeth are shown, the present invention is not so limited.

Finally, a locator tab 426 is provided on the upper surface of circular top wall 382, along the inner surface of gear teeth 424.

A mouthpiece 440, as shown in FIGS. 3, 4 and 40, is secured to the upper end of swirl nozzle 380. As shown in FIGS. 40-44, mouthpiece 440 includes a generally rectangular top wall 442 with an annular side wall 444 depending downwardly from the periphery of top wall 442. Because top wall 442 has a generally rectangular configuration and because of the annular configuration of side wall 444, upper portions at opposite sides 446 and 448 of side wall 444 corresponding to the lengthwise sides of top wall 442 slope upwardly in a converging manner toward each other. The lips of a user of the device are placed on sides 446 and 448 during inhalation. Of course, since the user's mouth is placed over mouthpiece, the various edges thereof are rounded.

A central opening 450 is centrally formed in top wall 442, and an annular connecting tube 452 is formed at the lower surface of top wall 442 in surrounding relation to opening 450. When mouthpiece 440 is seated on swirl nozzle 380, connecting tube 452 receives the upper end of supply chimney 404 of swirl nozzle 380 therein.

In order to secure mouthpiece 440 to swirl nozzle 380, the lower end of side wall 444 has a circular or annular shape. At the inner surface of this lower end of side wall 444, there is formed an annular V-shaped projection 454 which extends inwardly in the radial direction.

When mouthpiece 440 is positioned on swirl nozzle 380 and pressed down thereon, annular lip 422 of swirl nozzle 380, due to resilience of the plastic pieces, rides over V-shaped projection 454, so that V-shaped projection 454 retains annular lip 422, and thereby mouthpiece 440, on swirl nozzle 380. In such position, the lower edge of side wall 444 sits on annular ledge 420 of swirl nozzle 380.

Further, two sets of three gear teeth 460 are formed on the inner surface of diametrically opposite sides of annular side wall 444, immediately above annular V-shaped projection 454 and positioned centrally of opposite sides 446 and 448 of side wall 444. When mouthpiece 440 is assembled with swirl nozzle 380, gear teeth 460 engage with gear teeth 424 to prevent relative rotation between mouthpiece 440 and swirl nozzle 380.

Referring now to FIGS. 45-47, a closure cap 520 of metered powder dose dispenser 10 is provided as a closure for mouthpiece 440, and at the same time, functions to prime metered powder dose dispenser 10 for use. Specifically, closure cap 520 includes an upper elongated annular covering wall 522 which is closed at its upper end by a generally circular top wall 524. A lower annular securing skirt 526 of a larger diameter than annular covering wall 522, is secured to the lower end of annular covering wall 522 through an annular frusto-conical connector 528. The lower end of annular securing skirt 526 is open. Further, the inner diameter of lower annular securing skirt 526 is slightly larger than the outer diameter of upper annular wall 332 of adapter 320 so as to fit thereover.

In order to secure closure cap 520 onto metered powder dose dispenser 10, and particularly, in covering relation to mouthpiece 440, helix cams 530, preferably three spaced-apart, are formed on the inner surface of lower annular securing skirt 526. Thus, when closure cap 520 is inserted over powder housing 20, swirl nozzle 380 and mouthpiece 440, cams 530 of closure cap 520 initially vertically drop in entries 351 and then threadedly engage with double helical cam track 352 of adapter 320, until the lower edge of lower annular securing skirt 526 seats on the annular frusto-conical connecting section 334 of adapter 320.

It is noted that cams 530 and cam track 352 are provided in place of conventional screw threads. This is because, with conventional screw threads, cap 520 may be prematurely pulled off due to the tolerance of the threads. As a result, metered powder dose dispenser 10 may not be operated correctly, that is, not turned a full rotation (preferably about 120 degrees) during priming and delivery thereof. However, with cams 530 and cam track 352 having walls 353 of a square cross-section, numerous advantages are achieved, including preventing premature opening of cap 520, ease of use, ensuring proper location at all times of the rotational positions of the parts of dispenser 10, and ensuring that the counter (described hereinafter) is always correctly activated to always correctly change the dose count. Thus, cap 520 can not engage with adapter 320 until cams 530 are fully engaged in cam track 352.

It will be appreciated that the outer diameter of lower annular securing skirt 526 is substantially identical with the outer diameter of lower annular wall 322 of adapter 320 to provide a relative smooth, continuous appearance. In order to aid in the removal and closing of closure cap 520, the outer surface of lower annular securing skirt 526 is formed with a gripping surface 532 formed by undulations, knurling or the like, to enhance the gripping and rotating of closure cap 520.

As discussed above, closure cap 520 also serves to prime metered powder dose dispenser 10 for use. Specifically, three spaced-apart pairs of parallel, axially extending, spaced apart priming ribs 534 are formed on the inner surface of closure cap 520, extending a small distance down from frusto-conical connector 528 onto lower annular securing skirt 526. It is preferred that the priming ribs 534 be equally spaced-apart on the inner surface of closure cap 520. The priming ribs 534 of each pair are spaced apart by a distance slightly less than the width of driving openings 164, 166, 169, respectively, of driving body 120, for biasing spring fingers 161, 163, 165 inwardly, and also, for engaging sides of driving openings 164, 166 to rotate driving body 120. As shown best in FIG. 46, each of the priming ribs 534 has a lower ramp portion 535 and an upper ramp portion 537 which meet at an intermediate projecting portion 539 and reduce in thickness as they move away from projecting portion 539.

When closure cap 520 is removed from metered powder dose dispenser 10, metered dose holes 184, 184′ are in alignment with venturi conduits 64, 64′ ready for inhalation by the user. Thus, dispenser 10 is fully primed and ready for inhalation by a person. At such time, spring fingers 161, 163, 165 are positioned in recesses 340, 341, 342 of adapter 320. Thus, dispenser 10 is locked in this position.

The operation of inserting closure cap 520 is shown in FIGS. 48A-48E and FIGS. 49A and 49B. After the inhalation operation, closure cap 520 is positioned on the assembly, as shown in FIG. 48A. At this time, cams 530 are not engaged within cam tracks 352. Upon turning of closure cap 520, cams 530 fall within the beginning portions of cam tracks 352 and can be pushed down therein, as shown in FIGS. 48B and 48C. At this time, priming ribs 534 engage and push in spring fingers 161, 163, 165, and also engage sides of driving openings 164, 169. In other words, during the initial closure operation, lower ramp portions 535 of priming ribs 534 engage upper portions of spring fingers 161, 163, 165 and bias the same inwardly of recesses 340, 341, 342. This is shown in more detail in FIG. 49A. As a result, driving body 120 can rotate relative to adapter 320 to the closed position, as shown in FIGS. 48D and 48E. During this time, cap 520 engages with driving body 120, so that continued turning of cap 520 results in turning of driving body 120 relative to adapter 320. As cap 520 is rotated, it is pulled down by cams 530 riding in cam tracks 352.

At the completion of the rotation, and because of the configuration of spring fingers 161, 163, 165 and the complementary configuration of priming ribs 534, spring fingers 161, 163, 165 spring back into a locking position into mating engagement with priming ribs 534, 120 degrees offset from the inhalation position, that is, with spring fingers 161, 163, 165 positioned in recesses 340, 341, 342. Further, because of the mating relation of spring fingers 161, 163, 165 with priming ribs 534, priming ribs 534 are also, at this time, positioned in recesses 340, 341, 342. In other words, intermediate projecting portions 539 of priming ribs 534 are received within corresponding concave portions of spring fingers 161, 163, 165, as shown best in FIG. 49B.

It will be appreciated that when cap 520 is in the fully closed position of FIG. 48E, spring fingers 161, 163, 165 are returned to a free state, that is, a state in which there is no stress on spring fingers 161, 163, 165. This is provided so that over time, spring fingers 161, 163, 165 do not take a permanent set or deformation in a biased state, as with most plastic materials. This would be detrimental to the operation of the inhaler. The particular shapes of spring fingers 161, 163, 165 and priming ribs 534 are provided for this purpose.

Thus, closing rotation of closure cap 520 causes the rotation of driving body 120, and thereby of venturi conduits 64, 64′ relative to metered dose holes 184, 184′, to the stored position, 120 degrees out of alignment. During this travel, powder 62, 62′ is scraped into metered dose holes 184, 184′, so that metered powder dose dispenser 10 is primed.

When the user is ready to use metered powder dose dispenser 10, closure cap 520 is unscrewed from adapter 320. During such movement, spring fingers 161, 163, 165 initially engage with bevels 345 on recesses 340, 341, 342 which cause spring fingers 161, 163, 165 to move inwardly in order not to hinder rotation. Thereafter, as cap 520 begins to rise, spring fingers 161, 163, 165 again are engaged by priming ribs 534 which push in spring fingers 161, 163, 165. In other words, during the initial opening operation, upper ramp portions 537 of priming ribs 534 engage upper portions of spring fingers 161, 163, 165 and bias the same inwardly of recesses 340, 341, 342. Accordingly, driving body 120 can rotate relative to adapter 320 to the open position.

This results in opposite rotation of driving body 120, and thereby of venturi conduits 64, 64′ relative to metered dose holes 184, 184′ to a position in alignment. Thus, as soon as closure cap 520 is removed, metered dose holes 184, 184′ which are filled with powder 62, 62′, respectively, are in alignment with venturi conduits 64, 64′ and ready for inhalation. There is thus no need to provide any additional priming and set-up operation after closure cap 520 is removed.

With reference to FIG. 20, it is preferred that the powders 62, 62′ be kept out of contact. Accordingly, it is preferred that the metered dose holes 184, 184′ define fixed paths, F1, F2 during relative movement of the metering dose plate 180, back and forth to prime and administer a dose. It is preferred that the fixed paths F1, F2 sweep across an angle α in the range from about 90 to about 150 degrees, more preferably about 120 degrees. In addition, it is preferred that the fixed paths F1, F2 be spaced apart so that powder residue left along the fixed paths F1, F2 by one of the metered dose holes 184, 184′ not come into contact with the other metered dose hole 184, 184′. Accordingly, it is preferred that the fixed paths be separated between ends by an angle β in the range from about 30 to about 90 degrees, more preferably about 60 degrees. With the preferred arrangement equal spacing may be provided between the fixed paths F1, F2; i.e., the angle β is equal on both sides of the fixed paths F1, F2.

As will be appreciated by those skilled in the art, additional metered dose holes, e.g., three metered dose holes, may be utilized in accordance with the subject invention. The number of corresponding parts, e.g., venturi conduits, supply conduits, may require corresponding scaling up. With multiple metered dose holes, it is preferred that spacing be provided between any fixed paths that are defined. For example, with respect to the use of three metered dose holes, the angle α may be 90 degrees and the angle β may be 45 degrees. With this arrangement, equal spacing between three fixed paths may be achieved.

Further, closure cap 520 includes six equiangularly spaced protrusions 538 formed at the inner surface of covering wall 522, spaced a small distance from top wall 524.

A desiccant can be used with dispenser 10. A desiccant holder such as that disclosed in U.S. Pat. No. 6,240,918 may be utilized.

A counter mechanism 580 is provided for counting the number of doses that have been dispensed or indicating the number of doses that remain to be dispensed, so as to warn the user of impending powder depletion. Many types of mechanical and electrical counters are useful. A digital electronic counter can be disposed within the base or other areas of the device, and will require electrically conductive contacts which complete a circuit at some point in the dose loading operation; the characteristics of the required battery will be a factor in establishing a shelf life for the device. Presently preferred is counter mechanism 580, a decrementing mechanical counter that indicates the number of doses remaining to be dispensed.

Counter mechanism 580 is comprised of the aforementioned first and second rotation prevention spring detents 224 and 232 on base 200, the aforementioned transparent plastic window 330 of adapter 320, a continuous counter ring 590, an intermittent counter ring 620 and a spring-biased pawl assembly 640.

As shown in FIGS. 3, 4 and 50-53, continuous counter ring 590 is formed by a disc 592 having a wall with a substantially rectangular cross-section. An outer annular ledge 594 is formed on the outer, upper edge of disc 592 by cutting away disc 592 thereat. Further, a lower annular lip 596 axially extends from the lower, outer edge of disc 592, as a smooth extension of disc 592, but of a lesser cross-sectional width. As a result, an inner annular ledge 598 is formed at the lower edge of disc 592. In this regard, continuous counter ring 590 can be seated on base 200, and in particular, inner annular ledge 598 seats upon circular top wall 202 of base 200 and lower annular lip 596 seats on annular ledge 206 of base 200 in surrounding relation to circular top wall 202.

A plurality of numerical indicia 600 are printed on the smooth combined outer surface of disc 592 and lower annular lip 596. Specifically, two successive sets of numbers “0” through “9” are printed equiangularly thereabout. Numerical indicia 600 are printed in a vertical manner. Thus, indicia 600 can be read while metered powder dose dispenser 10 is upright, that is, in the manner that it should be used.

Twenty gear teeth 602 are equiangularly formed on the inner surface of disc 592 in correspondence with the twenty numbers of numerical indicia 600. All gear teeth 602 have the same depth in the radial direction, with the exception that diametrically opposite gear teeth 604 and 606 of gear teeth 602, corresponding to the opposite numbers “5” of numerical indicia 600, are deeper than the remaining gear teeth 602, that is, gear teeth 604 and 606 extend outwardly in the radial direction to a greater extent than the remaining gear teeth 602. When continuous counter ring 590 is seated on base 200, first rotation prevention spring detent 224 of base 200 engages with one gear tooth 602 at a time, to prevent clockwise rotation of continuous counter ring 590 on base 200.

As shown in FIGS. 3, 4 and 54-57, intermittent counter ring 620 is formed by a disc 622 having a wall with a substantially rectangular cross-section. A lower annular lip 624 axially extends from the lower, outer edge of disc 622, as a smooth extension of disc 622, but of a lesser cross-sectional width. As a result, an inner annular ledge 626 is formed at the lower edge of disc 622. In this regard, intermittent counter ring 620 can be rotatably seated on continuous counter ring 590, and in particular, inner annular ledge 626 is spaced above continuous counter ring 590, while lower annular lip 624 seats on outer annular ledge 594 of continuous counter ring 590.

A plurality of numerical indicia 628 are printed on the smooth combined outer surface of disc 622 and lower annular lip 624. Specifically, numbers “0” through “19” are printed equiangularly thereabout. Numerical indicia 628 are printed in a vertical manner. Thus, indicia 628 can be read while metered powder dose dispenser 10 is upright, that is, in the manner that it should be used.

Twenty gear teeth 630 are equiangularly formed on the inner surface of disc 622 in correspondence with the twenty numbers of numerical indicia 628. All gear teeth 630 have the same depth in the radial direction. When intermittent counter ring 620 is seated on continuous counter ring 590, second rotation prevention spring detent 232 of base 200 engages with one gear tooth 630 at a time, to prevent clockwise rotation of intermittent counter ring 620 on base 200. As will be appreciated from the discussion which follows, gear teeth 630 extend along a larger diameter circle than gear teeth 602, so that gear teeth 630 are outwardly displaced in the radial direction from gear teeth 602.

Further, a dose limiting tab 632 extends upwardly from the upper surface of disc 622, corresponding to a position between numbers “9” and “10”, to prevent operation of metered powder dose dispenser 10 after a prescribed number of doses have been dispensed. For example, where metered powder dose dispenser 10 is limited to dispensing 200 doses, dose limiting tab 632 can abut against a dosage limiter tab 336 of adapter 320 after dispensing of the two hundredth dose, to prevent further relative rotation of powder housing 20 with respect to metering dose plate 180, as will be described with respect to the operation hereinafter.

Initially, number “19” of indicia 628 is aligned with number “9” of indicia 600 to form the number 199, which is exposed through transparent plastic window 330 of adapter 320. After the first dose is dispensed, only continuous counter ring 590 rotates so that the numbers “19” and “8”, respectively, are exposed to form the number “198” which is exposed through window 330. After the next nine doses, only continuous counter ring 590 rotates one increment at a time for each dose. After the number “190” is exposed through window 330, the next dose results in both continuous counter ring 590 and intermittent counter ring 620 rotating to form the number “189”. This operation continues until the number “00” is exposed through window 330. At this time, intermittent counter ring 620 has been rotated to a position so that dose limiting tab 632 abuts against dosage limiter tab 336 of adapter 320, to prevent further relative rotation of powder housing 20 with respect to metering dose plate 180.

In order to cause such rotation of continuous counter ring 590 and intermittent counter ring 620, spring-biased pawl assembly 640 includes a pawl driver 642, as shown in FIGS. 3, 4 and 58-62. Pawl driver 642 includes an arcuate outer wall 644 having a height greater than the combined height of continuous counter ring 590 and intermittent counter ring 620. A U-shaped retainer 650 is connected to the free ends of arcuate wall 644. U-shaped retainer 650 has a height less than that of arcuate wall 644. Accordingly, a loop defining an open area 652, is formed by arcuate wall 644 and U-shaped retainer 650. A flange 648 of a substantially triangular cross-sectional configuration, forms an extension at one side of arcuate wall 644 at the intersection thereof with U-shaped retainer 650, but being of a height substantially equal to that of U-shaped retainer 650.

A pawl 654 is centrally formed on the outer or convex surface of arcuate wall 644. Thus, when pawl driver 642 is inserted on circular top wall 202 of base 200 in surrounding relation to cylindrical boss 216, pawl 654 can be inserted within a gear tooth 602. However, because gear teeth 630 extend along a larger diameter circle than gear teeth 602, pawl 654 can only engage with gear teeth 602 and not with gear teeth 630. The only exception is when pawl 654 engages within one of gear teeth 604 or 606. In such case, because gear teeth 604 and 606 are deeper than the remaining gear teeth 602, pawl 654 can reach into and engage with gear teeth 630. Since gear teeth 604 and 606 are spaced apart by ten gear teeth, pawl 654 engages within one of the gear teeth 604 or 606 every tenth dose dispensing, and thereby engages within one of gear teeth 630 at such time to rotatably drive intermittent counter ring 620 with continuous counter ring 590.

In order to bias pawl 654 into engagement with gear teeth 602, a bent, substantially inverted L-shaped spring 658 has one end integrally formed centrally, in regard to the widthwise and heightwise directions, at the inner surface of arcuate wall 644, with the free end thereof hanging down to push against cylindrical boss 216 of base 200 within radial segment 219, thereby biasing pawl assembly 640 outwardly in the radial direction. This causes pawl 654 to enter into engagement with gear teeth 602.

It will be appreciated that, by forming spring 658 integrally in a single molding operation with pawl assembly 640, the number of parts is reduced, a single molding operation is utilized, assembly of the parts is easier, and the spring can be made more flexible and reliable.

It will be appreciated that, when pawl assembly 640 is positioned on base 200, opposite sides of U-shaped retainer 650 are positioned within angled stub walls 221 and 223, so that there is just sufficient room for pawl assembly 640 to rotate by a small angle, in order to function as a ratchet assembly with respect to the gear teeth of counter rings 590 and 620.

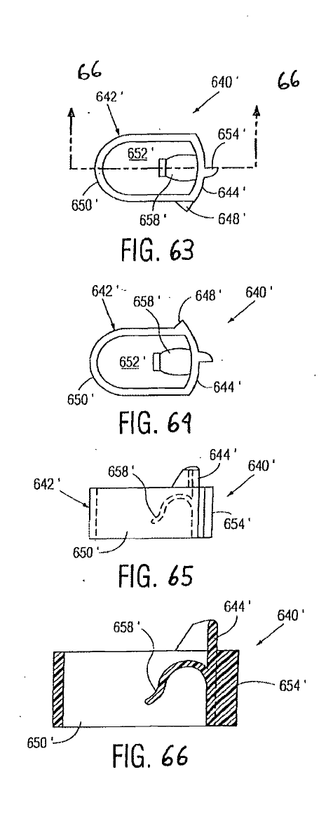

Referring to FIGS. 63-66, there is shown a spring-biased pawl assembly 640′ according to another embodiment of the present invention, in which elements corresponding to those of pawl assembly 640 of FIGS. 58-62 are identified by the same reference numerals, with a prime (′) added thereto.

The only difference between pawl assembly 640′ and pawl assembly 640 is that the free end of spring 658′ of pawl assembly 640′ has a slight convex curvature away from the fixed end thereof.

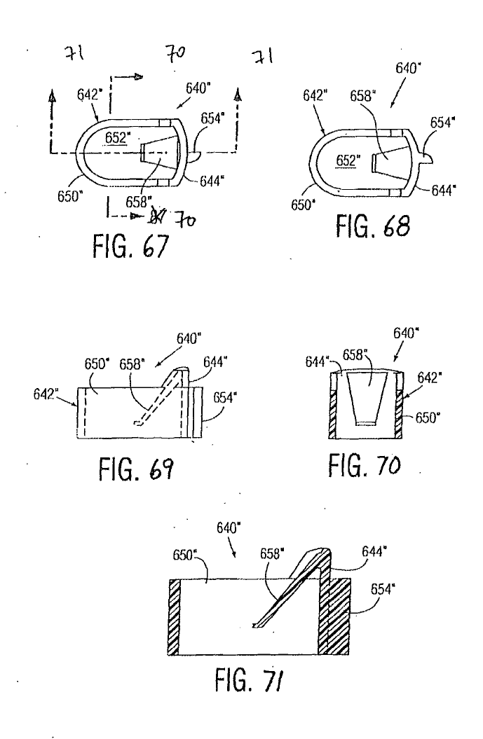

Referring to FIGS. 67-71, there is shown a spring-biased pawl assembly 640″ according to still another embodiment of the present invention, in which elements corresponding to those of pawl assembly 640 of FIGS. 58-62 are identified by the same reference numerals, with a double prime (″) added thereto.

One difference between pawl assembly 640″ and pawl assembly 640 is that spring 658′ of pawl assembly 640″, rather than being formed as a substantially L-shaped member, is formed is a generally linear member with tapered sides, extending at an angle from the upper end of the inner surface of arcuate wall 644″. Another difference is that flange 648 is eliminated entirely.

In the operation of counter mechanism 580, lower spring retainer 260 rotates 120 degrees with reservoir body 22 relative to metering dose plate 180 between the stored position when closure cap 520 is threaded onto adapter 320 and the inhalation position when closure cap 520 is removed from adapter 320. When metered powder dose dispenser 10 is in the stored position, pawl 654 is engaged within a shallow gear tooth 602 of continuous counter ring 590, and therefore, does not engage with a gear tooth 630. Further, in such position, pawl driving end 276 of arcuate pawl driving wall 274 engages with pawl assembly 640.

When reservoir body 22 is rotated the first 118 degrees toward the inhalation position, pawl driving end 278 of arcuate pawl driving wall 274 is rotated into engagement with the opposite side of pawl assembly 640. As a result, pawl 654 is rotated so that it rides out of the shallow gear tooth 602, thereby compressing spring 658. When ten doses have been dispensed, continued rotation to the full 120 degrees causes pawl 654 to rotate a slight amount and fall into the next gear tooth 604, which is a deep gear tooth, for example. Specifically, spring 658 biases pawl 654 into gear tooth 604. Since gear tooth 604 is a deep gear tooth, pawl 654 also enters one of the gear teeth 630. At this point, metered powder dose dispenser 10 is in the inhalation position in which metered dose holes 184, 184′ are in alignment with venturi conduits 64, 64′.

After the user inhales the dose of powder 62, 62′, closure cap 520 is threaded back onto adapter 320. As a result, reservoir body 22 rotates back to its initial position, which also results in rotation of lower spring retainer 260. During this rotation back 120 degrees, that is, pawl driving end 276 of arcuate pawl driving wall 274 engages with pawl assembly 640 at the end of its movement to rotate pawl assembly 640 to its initial position. During such movement, since pawl 654 is engaged within deep gear tooth 604 and one of the gear teeth 630, both continuous counter ring 590 and intermittent counter ring 620 are rotated together one increment. In the case where pawl 654 is not engaged with one of the deep gear teeth 604 or 606, pawl does not engage with a gear tooth 630, so that only the continuous counter ring 590 would be rotated.

It will be appreciated that continuous counter ring 590 and intermittent counter ring 620 cannot rotate in the opposite direction because of first and second rotation prevention spring detents 224 and 232 which engage with gear teeth 602 and 630, respectively.

It will be appreciated that various changes can be made to the scope of the present invention. For example, rotation of metering dose plate 180 need not be 120 degree, but could be for a lesser or greater arcuate distance. In such case, the length of arcuate pawl driving wall 274 would be changed to incrementally drive pawl assembly 640.

Accordingly, with the present invention, a metered powder dose dispenser 10 is provided that accurately measures the doses of powdered medicament to be delivered to the patient. Specifically, dispenser 10 is greatly simplified in construction and assembly over the prior art.

All of the above elements, with the exception of metal plate 93′ and spring 290, are preferably fabricated from readily available plastics, while the former parts are preferably fabricated from suitable metals. Typically, the various components which do not require porosity or other special properties will be molded from one or more thermoplastic substances having the desired rigidity and strength. In some embodiments, the component containing the powder receptacle is relatively thin and, to maintain a required degree of surface flatness, will be constructed from a less easily deformed substance such as a reinforced plastic, ceramic or metal. Of course, materials selected must be chemically compatible with the medication to be dispensed. For reasons of cost, a maximum utilization of plastics will be preferred where the device is intended to be disposable with no, or only a limited number of, medicament refills after the initial charge has been dispensed. Other “composite” components can be used elsewhere in the device where special properties are required.

In order to assemble metered powder dose dispenser 10, powder housing 20 is first assembled. Specifically, reservoir plug 90 is inserted within reservoir body 22, swirl nozzle 380 is assembled with driving body 120 and mouthpiece 440 is assembled with swirl nozzle 380. Next, continuous counter ring 590 is fit onto base 200 and intermittent counter ring 620 is fit onto continuous counter ring 590. Both counter rings 590 and 620 are rotated until the number “19”, of intermittent counter ring 620 and the number “19” of continuous counter ring 590 are in alignment for display through window 330. In other words, this corresponds to the number “199”.

Pawl assembly 640 is then positioned on top circular wall 202 of base 200 in surrounding relation to cylindrical boss 216 and between stub walls 221 and 223, with pawl 654 being biased into engagement with gear tooth 604 in alignment with the number “5” and the gear tooth 630 in alignment with the number “5”, that is, in alignment with the number “5”. It will be appreciated that first and second rotation prevention spring detents 224 and 232 are in alignment with gear tooth 606 corresponding to number “0” and with the gear tooth 630 corresponding to the number “19”.

Thereafter, lower spring retainer 260 is positioned on boss 216 in surrounding relation to retaining post 218, with narrow driven ear 270 in alignment with the number “199” on rings 590 and 620. In such case, pawl driving end 276 thereof is in abutment with flange 648 of pawl assembly 640. Coil spring 290 is then seated on disc 262 of lower spring retainer 260, and support plate 300 is placed on top of coil spring 290, with narrow driven ear 306 thereof in alignment with narrow driven ear 270 of lower spring retainer 260. Then, annular mounting post 188 of metering dose plate 180 is positioned through central circular hole 310 of support plate 300 and over retaining post 218 of base 200, with bar 190 and slot 222 in alignment. In such case, metered dose holes 184, 184′ is in alignment with radially extending slot 312 of support plate 300.

Then, reservoir body 22, having reservoir plug 90 assembled therewith, is inserted over metering dose plate 180, support plate 300, coil spring 290 and lower support plate 260, such that narrow driven ears 270 and 306 fit within narrow drive slots 36, and wider driven ears 271, 272 and 307, 308 fit within wider drive slots 34, 35 of reservoir body 22. In such case, venturi conduits 64, 64′ are in alignment with metered dose hole 184. In order to assemble the above parts together, adapter 320 is then placed over the above assembly such that slot 326 thereof is in alignment with post 214 of base 200. Adapter 320 is then pressed down until annular ledge 210 of base 200 snaps into annular groove 324 of adapter 320. At this time, coil spring 290 is compressed, the number “199” appears through window 330 of adapter 320, and recesses 340, 341, 342 of adapter 320 are in alignment with drive slots 34, 35, 36, respectively, of reservoir body 22.

Thereafter, powder supply conduit 60 is filled through the upper open end thereof. Then, driving body 120, with nozzle 380 and mouthpiece 440 thereon, is fit over reservoir body 22, such that circular plug conduit 144 of driving body 120 plugs the upper open ends of powder supply conduits 60, 60′ and such that the upper open ends of venturi conduits 64, 64′ extend through circular openings 142, 142′ in driving body 120. In this position, the lower edge of lower annular skirt section 128 of driving body 120 is positioned immediately above the upper edge of upper annular wall 332 of adapter 320.

Closure cap 520 is then threaded onto adapter 320, whereby powder housing 20 is rotated 120 degrees relative to metering dose plate 180 so as to prime metered powder dose dispenser 10, that is, so as to scrape powder 62, 62′ into metered dose holes 184, 184′. This moves pawl 654 to the next gear tooth 602.

When a user desires to inhale a dosage of the powder 62, 62′, closure cap 520 is unthreaded and removed, thereby rotating powder housing 20 back 120 degrees so as to align venturi conduits 64, 64′ with metered dose holes 184, 184′, ready for inhalation. At this time, pawl 654 is rotated one increment, whereby the next number “198” is displayed through window 330. When all 200 doses have been used, dose limiting tab 632 of intermittent counter ring 620 abuts against dosage limiter tab 336 of adapter 320 to prevent further rotation for dispensing. Accordingly, the numbers will not continue from “00” to “199”.

Having described specific preferred embodiments of the invention with reference to the accompanying drawings, it will be appreciated that the present invention is not limited to those precise embodiments and that various changes and modifications can be effected therein by one of ordinary skill in the art without departing from the scope or spirit of the invention as defined by the appended claims. The term ‘comprising’ is defined as ‘including but not limited to’.

What is claimed is:

1. A powder dispenser comprising:
a first powder reservoir having at least one first outlet opening;
a second powder reservoir having at least one second outlet opening, the second outlet opening being spaced from the first outlet opening;
a metering dose plate having a first metered dose hole and a second metered dose hole, the first and second metered dose holes being configured to each hold a predetermined amount of powder,
wherein the metering dose plate is disposed adjacent to the first and second outlet openings, wherein, relative movement between the metering dose plate and the first and second outlet openings causes the metering dose plate to be selectively located at first and second positions relative to the first and second outlet openings,
wherein, with the metering dose plate moving relative to the first and second outlet openings from the first position to the second position, the first metered dose hole passes below the first outlet opening, and the second metered dose hole passes below the second outlet opening, and,
wherein, the first metered dose hole defining a fixed first path as the metering dose plate moves between the first and second positions relative to the first and second outlet openings, the second metered dose hole defining a second fixed path as the metering dose plate moves between the first and second positions relative to the first and second outlet openings, the first fixed path being spaced from the second fixed path such that the first metered dose hole does not overlap the second path during movement of the metering dose plate and such that the second metered dose hole does not overlap the first fixed path during movement of the metering dose plate.

2. A dispenser as in claim 1, wherein, relative to the center of the metering dose plate, the first fixed path extends across an angle of approximately 120 degrees.

3. A dispenser as in claim 2, wherein the second fixed path extends across an angle of approximately 120 degrees.

4. A dispenser as in claim 1, wherein the first fixed path is separated from the second fixed path by at least an angle of at least 60 degrees as measured relative to the center of the metering dose plate.

5. A dispenser as in claim 1 further comprising a first inhalation conduit, wherein, with the metering dose plate being in the first position, the first metered dose hole is axially aligned with the first inhalation conduit.

6. A dispenser as in claim 5 further comprising a second inhalation conduit, wherein, with the metering dose plate being in the first position, the second metered dose hole is axially aligned with the second inhalation conduit.

7. A dispenser as in claim 1, wherein the first metered dose hole is configured to hold substantially the same amount of powder as the second metered dose hole.

8. A dispenser as in claim 1, wherein the first metered dose hole is configured to hold a different amount of powder from the second metered dose hole.

9. A dispenser as in claim 1, wherein the metering dose plate is held stationary.

10. A drug product comprising a dry powder inhaler and at least one dose of at least one active pharmaceutical agent; wherein the dry powder inhaler comprises at least two reservoirs comprising the at least one dose.

11. The drug product of claim 10, wherein the at least two reservoirs separately comprise different active pharmaceutical agents.

12. The drug product of claim 10, the wherein when the dry powder inhaler is actuated, the at least one dose is emitted simultaneously from the at least two reservoirs.

13. The drug product of claim 10, wherein the dry powder inhaler can accommodate at least one dose of at least two incompatible active pharmaceutical agents.

14. A drug product comprising a dry powder inhaler and at least one dose of at least one active pharmaceutical agent; wherein the dry powder inhaler comprises at least two reservoirs separately comprising different active pharmaceutical agents and the at least two reservoirs comprises the at least one dose that are emitted simultaneously from the at least two reservoirs when the dry powder inhaler is actuated.

15. The drug product of claim 14, wherein the dry powder inhaler can accommodate at least one dose of at least two incompatible active pharmaceutical agents.

16. A powder dispenser comprising:
a first powder reservoir having at least one first outlet opening;
a second powder reservoir having at least one second outlet opening, the second outlet opening being spaced from the first outlet opening.
